SEQUENCE LISTING (2 sequences)

SEQ ID NO: 1 - DNA, 52 bp, Artificial Sequence
  siRNA hairpin RNA
gcagcagctgaacaacatgttcaagagacatgttgttcagctgctgcttttt

SEQ ID NO: 2 - DNA, 9 bp, Artificial Sequence
  siRNA hairpin RNA loop sequence
ttcaagaga

(19) United States
(12) Patent Application Publication
(10) Pub. No.: US 2009/0208534 A1
(43) Pub. Date: Aug. 20, 2009

(54) ATTENUATED SALMONELLA AS A DELIVERY SYSTEM FOR SIRNA-BASED TUMOR THERAPY

(75) Inventors: Deqi Xu, Colombia, MD (US); Dennis J. Kopecko, Silver Spring, MD (US); Jiadi Hu, Columbia, MD (US); Ling Zhang, Changchun City (CN); Xuejian Zhao, Changchun City (CN); Lifang Gao, Changchun City (CN)

Correspondence Address: KNOBBE, MARTENS, OLSON & BEAR, LLP, 2040 MAIN STREET, FOURTEENTH FLOOR, IRVINE, CA, 92614, US

(73) Assignee: The Government of The United States of America, as represented by the Secretary, Dept.of Health, Rockville, MD (US)

(21) Appl. No.: 12/374,916

(22) PCT Filed: Jul. 24, 2007
(86) PCT No.: PCT/US07/74272

§ 371 Date: Jan. 23, 2009

(30) Foreign Application Priority Data
Jul. 26, 2006 (CN) 200610017045.5

(51) Int. Cl. A61K 39/112; C12N 1/20; A61P 35/00

(52) U.S. Cl. 424/258.1; 435/252.8

(57) ABSTRACT

The invention relates to an attenuated Salmonella sp. that is capable of targeting a solid tumor when administered in vivo comprising a short hairpin (sh) RNA construct, and methods of inhibiting the growth or reducing the volume of a solid tumor cancer comprising administering an effective amount of an attenuated Salmonella sp. to a patient having a solid tumor cancer, wherein said attenuated Salmonella sp. is a tumor targeting attenuated Salmonella sp. expressing a short hairpin (sh) RNA which attenuated Salmonella sp. is capable of inhibiting the growth or reducing the volume of the solid tumor cancer when administered in vivo.

### BACKGROUND OF THE INVENTION

RNA interference (RNAi) is an evolutionarily conserved, posttranscriptional gene-silencing mechanism wherein a small interfering double-stranded RNA (siRNA) directs a sequence-specific degradation of its target mRNA (Hannon, G. J. 2002 Nature 418:244-251). Because of their unparalleled target specificity, there has been an intensive effort to use siRNAs as therapeutics for various diseases, especially for cancer therapy. Because synthetic siRNAs can only transiently decrease the target gene expression in proliferating cancer cells (Tuschl, T. and Borkhardt, A. 2002 Mol Interv 2:158-167), a sustained, localized supply of anticancer siRNAs is critical for imparting a strong therapeutic benefit. Plasmid-based expression of gene-specific small hairpin RNAs (shRNA), under the control of RNA polymerase III-dependent promoters (e.g., U6 and H1), produces a sustainable and economical source of siRNAs for therapeutic purposes. The shRNAs are processed intracellularly by the enzyme Dicer into siRNAs. Some groups have reported successful application in vivo following systemic administration with shRNA-encoding plasmid DNA (Spankuch, B. et al. 2004 J Natl Cancer Inst 96:862-872; Takeshita, F. and Ochiya, T. 2006 Cancer Sci 97:689-696; Xiang, S. et al. 2006 Nat Biotechnol 24:697-702). Unfortunately, in most of these approaches, the therapeutics do not reach the tumors in effective doses, or distribution to unwanted sites and degradation by nucleases result in limited antitumor effect. The success of siRNAs as cancer therapeutics relies on the development of safe, economical, and efficacious in vivo delivery systems into tumor cells. Although siRNAs can be used as therapeutics in vivo, their intratumoral delivery, specifically across the plasma membrane of cells, is not achieved easily. Furthermore, they are ineffective at killing quiescent tumor cells that are distantly located from the vasculature and metastatic tumors because of their heterogeneous microenvironments. The ideal delivery system would be (a) nontoxic to normal cells and (b) able to deliver the therapeutic efficiently and specifically to the tumor.

The discovery that genes vectored by bacteria can be functionally transferred to mammalian cells has suggested the possible use of bacterial vectors as vehicles for gene therapy. Genetically modified, nonpathogenic bacteria have been used as potential antitumor agents, either to elicit direct tumoricidal effects or to deliver tumoricidal molecules (Clairmont, C. et al. 2000 J Infect Dis 181:1996-2002; Bermudes, D. et al. 2002 Curr Opin Drug Discov Devel 5:194-199; Zhao, M. et al. 2005 Proc Natl Acad Sci USA 102:755-760; Zhao, M. et al. 2006 Cancer Res 66:7647-7652). Bioengineered attenuated strains of Salmonella enterica serovar typhimurium (S. typhimurium) have been shown to accumulate preferentially >1,000-fold greater in tumors than in normal tissues and to disperse homogeneously in tumor tissues (Pawelek, J. et al. 1997 Cancer Res 57:4537-4544; Low, K. B. et al. 1999 Nat Biotechnol 17:37-41). Preferential replication allows the bacteria to produce and deliver a variety of anticancer therapeutic agents at high concentrations directly within the tumor, while minimizing toxicity to normal tissues. These attenuated bacteria have been found to be safe in mice, pigs, and monkeys when administered i.v. (Zhao, M. et al. 2005 Proc Natl Acad Sci USA 102:755-760; Zhao, M. et al. 2006 Cancer Res 66:7647-7652; Tjuvajev J. et al. 2001 J Control Release 74:313-315; Zheng, L. et al. 2000 Oncol Res 12:127-135), and certain live attenuated Salmonella strains have been shown to be well tolerated after oral administration in human clinical trials (Chatfield, S, N. et al. 1992 Biotechnology 10:888-892; DiPetrillo, M. D. et al. 1999 Vaccine 18:449-459; Hohmann, E. L. et al. 1996 J Infect Dis 173:1408-1414; Sirard, J. C. et al. 1999 Immunol Rev 171:5-26). The S. typhimurium phoP/phoQ operon is a typical bacterial two-component regulatory system composed of a membrane-associated sensor kinase (PhoQ) and a cytoplasmic transcriptional regulator (PhoP: Miller, S. I. et al. 1989 Proc Natl Acad Sci USA 86:5054-5058; Groisman, E. A. et al. 1989 Proc Natl Acad Sci USA 86: 7077-7081). phoP/phoQ is required for virulence, and its deletion results in poor survival of this bacterium in macrophages and a marked attenuation in mice and humans (Miller, S. I. et al. 1989 Proc Natl Acad Sci USA 86:5054-5058; Groisman, E. A. et al. 1989 Proc Natl Acad Sci USA 86: 7077-7081; Galan, J. E. and Curtiss, R. III. 1989 Microb Pathog 6:433-443; Fields, P. I. et al. 1986 Proc Natl Acad Sci USA 83:189-193). phoP/phoQ deletion strains have been employed as effective vaccine delivery vehicles (Galan, J. E. and Curtiss, R. III. 1989 Microb Pathog 6:433-443; Fields, P. I. et al. 1986 Proc Natl Acad Sci USA 83:189-193; Angelakopoulos, H. and Hohmann, E. L. 2000 Infect Immun 68:213-241). More recently, attenuated Salmonellae have been used for targeted delivery of tumoricidal proteins (Bermudes, D. et al. 2002 Curr Opin Drug Discov Devel 5:194-199; Tjuvajev J. et al. 2001 J Control Release 74:313-315).

### SEGUE TO THE INVENTION

We report here the use of an attenuated phoP/phoQ null S. typhimurium as a delivery system for siRNA-based tumor therapy.

### SUMMARY OF THE INVENTION

In one embodiment, the invention relates to an attenuated Salmonella sp. that is capable of targeting a solid tumor when administered in vivo comprising a short hairpin (sh) RNA construct.

In another embodiment, the invention relates to a method of inhibiting the growth or reducing the volume of a solid tumor cancer comprising administering an effective amount of an attenuated Salmonella sp. to a patient having a solid tumor cancer, wherein said attenuated Salmonella sp. is a tumor targeting attenuated Salmonella sp. expressing a short hairpin (sh) RNA which attenuated Salmonella sp. is capable of inhibiting the growth or reducing the volume of the solid tumor cancer when administered in vivo.

### BRIEF DESCRIPTION OF THE DRAWINGS

FIG. 1. (A) structure of pSi-Stat3 plasmid containing the sequence of Stat3-specific hairpin RNA (shRNA-Stat3; arrow). (B) expression of GFP of pSi-Stat3 and pSi-Scramble in stable infected RM-1 cells versus mock uninfected cells. Magnification, ×400.

FIG. 2. shRNA-mediated knockdown of STAT3 expression. Northern (A) and Western (C) blot analyses of Stat3 expression. Equal amounts of total RNA (20 μg) were used for Northern blot analysis. (B) quantification of Stat3 mRNA from three separate experiments and normalized to that of β-actin. *, P<0.01 versus mock and scrambled vector control. (D) quantification of Stat3 protein levels.

FIG. 3. Si-Stat3 inhibits cell growth and induces apoptosis. (A) cells were stained with AnnCy3 (dark gray) and 6-CF (light gray) to visualize apoptotic cells using confocal microscopy. Live cells were labeled only with 6-CF (light gray); necrotic cells were labeled only with AnnCy3 (dark gray); and cells undergoing apoptosis were double labeled yielding a white shade in merged images. (B) MTT assays. Points, mean of three separate experiments. *, P<0.01 versus mock and pSi-Scramble. Tumor cell viability (A value) was significantly reduced by treatment with pSi-Stat3. P<0.05 (n=3). (C) expression of Bcl-2, cyclin D1, c-Myc, and VEGF proteins as revealed by Western blot analyses. Mock, untreated cells. (D) quantification of the images in (C).

FIG. 4. Effects of systemically administered recombinant S. typhimurium on prostate tumor growth in vivo. (A) representative mice treated with recombinant bacteria carrying various plasmids after orthotopic implantation of prostate tumor. Note a significant loss of tumor volume in mice treated with Salmonella-pSi-Stat3 compared with the control. Tumor locations (arrows) (B) immunohistochemical analyses of Stat3 and Ki-67 expression. Note a strong positive staining for Stat3 and Ki-67 in pSi-Scramble-treated tumor, in sharp contrast to those treated with Si-Stat3. Magnification, ×400. (C) H&E staining and TUNEL (magnification, ×200) of tumors. TUNEL-positive cells (dark).

FIG. 5. MMP-2 activity in RM-1 cells. A) Western blot analysis of MMP-2, B) Quantification of the images in (A).

FIG. 6. Survival curves of mice injected with Salmonella-pSi-Stat3.

FIG. 7. Recombinant bacterial distribution in C57BL6 tumor-bearing mice. A) colony forming units (cfu)/g tissue for tumor, liver and spleen, B) Bacterial distribution in tissue sections of liver, spleen and tumor.

FIG. 8. Mechanisms of RNA interference in mammalian cells.

FIG. 9. RISC loading and activation.

FIG. 10. RNA interference effector molecules. A) Synthetic siRNAs, B) Expressed shRNAs.

FIG. 11. Properties of STAT molecules.

### DETAILED DESCRIPTION OF THE PREFERRED EMBODIMENT

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3rd ed., J. Wiley & Sons, Chichester, N.Y., 2001.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

According to the present invention, attenuated Salmonella are advantageously used in methods to produce a tumor growth inhibitory response or a reduction of tumor volume in an animal including a human patient having a solid tumor cancer. For such applications, it is advantageous that the attenuated Salmonella possess tumor targeting ability or target preferably to tumor cells/tissues rather than normal cells/tissues. Additionally, it is advantageous that the attenuated Salmonella possess the ability to retard or reduce tumor growth and/or express a short hairpin RNA that retards or reduces tumor growth. Tumor targeting ability can be assessed by a variety of methods known to those skilled in the art, including but not limited to cancer animal models.

When administered to a patient, e.g., an animal for veterinary use or to a human for clinical use, the attenuated Salmonella can be used alone or may be combined with any physiological carrier such as water, an aqueous solution, normal saline, or other physiologically acceptable excipient. In general, the dosage ranges from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg.

The attenuated Salmonella of the present invention can be administered by a number of routes, including but not limited to: orally, intranasally, topically, injection including, but limited to intravenously, intraperitoneally, subcutaneously, intramuscularly, intratumorally, i.e., direct injection into the tumor, etc.

Intratumoral Delivery and Suppression of Prostate Tumor Growth by Attenuated Salmonella enterica Serovar typhimurium Carrying Plasmid-Based Small Interfering RNAs

The facultative anaerobic, invasive Salmonella enterica serovar typhimurium (S. typhimurium) has been shown to retard the growth of established tumors. We wondered if a more effective antitumor response could be achieved in vivo if these bacteria were used as tools for delivering specific molecular antitumor therapeutics. Constitutively activated transcription factor signal transducer and activator of transcription 3 (STAT3) promotes the survival of a number of human tumors. In this study, we investigated the relative efficacies of attenuated S. typhimurium alone or combined with Stat3-specific small interfering RNA (siRNA) in terms of tumor growth and metastasis. The bacteria preferentially homed into tumors over normal liver and spleen tissues in vivo. S. typhimurium expressing plasmid-based Stat3-specific siRNAs significantly inhibited tumor growth, reduced the number of metastastic organs, and extended the life time for C57BL6 mice bearing an implanted prostate tumor, versus bacterial treatment alone. These results indicate that attenuated S. typhimurium combined with an RNA interference approach serve as the basis for the treatment of primary as well as metastatic cancer.

Construction of shRNA Expression Vectors and Cell Infection

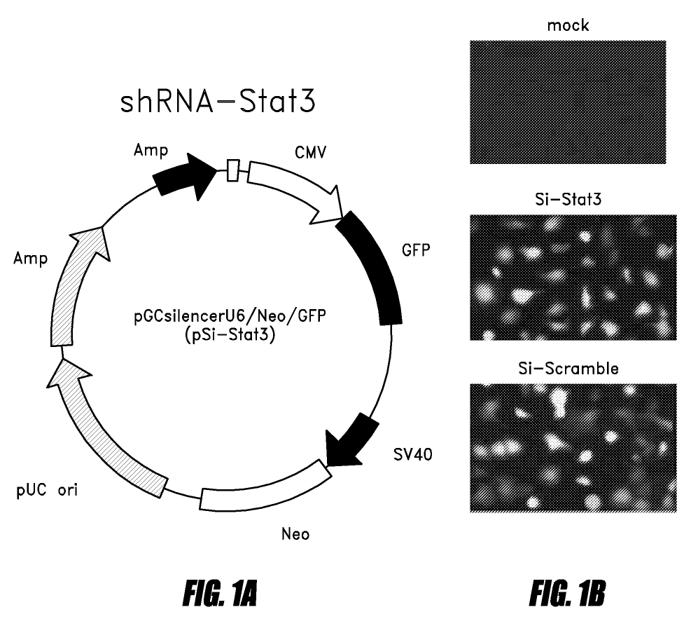

To show the utility of attenuated S. typhimurium-carried, Stat3specific siRNA for tumor therapy, we first generated plasmid vectors that express a Stat3-specific siRNA (Si-Stat3) and a control scrambled siRNA (Si-Scramble). Because of its potent antitumor effects, the target for Si-Stat3 was chosen from the SH2 domain of human Stat3 based on our earlier study (Gao, L. et al. 2005 Clin Cancer Res 11:6333-6341). Synthetic oligonucleotides (20 bp) capable of coding for the Si-Stat3 and Si-Scramble siRNAs were cloned into pGCsilencerU6/Neo/GFP, a plasmid containing the GFP gene. The resultant plasmids pSi-Stat3 and pSi-Scramble were transformed into S. typhimurium and used for transfection into RM-1 cells (FIG. 1A). Virtually identical transfection efficiencies were observed for each plasmid as determined by the expression of GFP in RM-1 cells (FIG. 1B).

In this study, the invasive recombinant S. typhimurium carrying either the pSi-Stat3 or pSi-Scramble plasmids were directly cocultured with a mouse prostate carcinoma cell line (RM-1), and stable cell lines RM-Si-Stat3 and RM-Si-Scramble were established after G418 selection. The continued expression of GFP, in the absence of bacteria in the cell lines, indicates that the siRNA expression vectors were stably integrated into the host cell genome.

Effects of Bacterially Delivered shRNAs on Cell Growth and Cycling

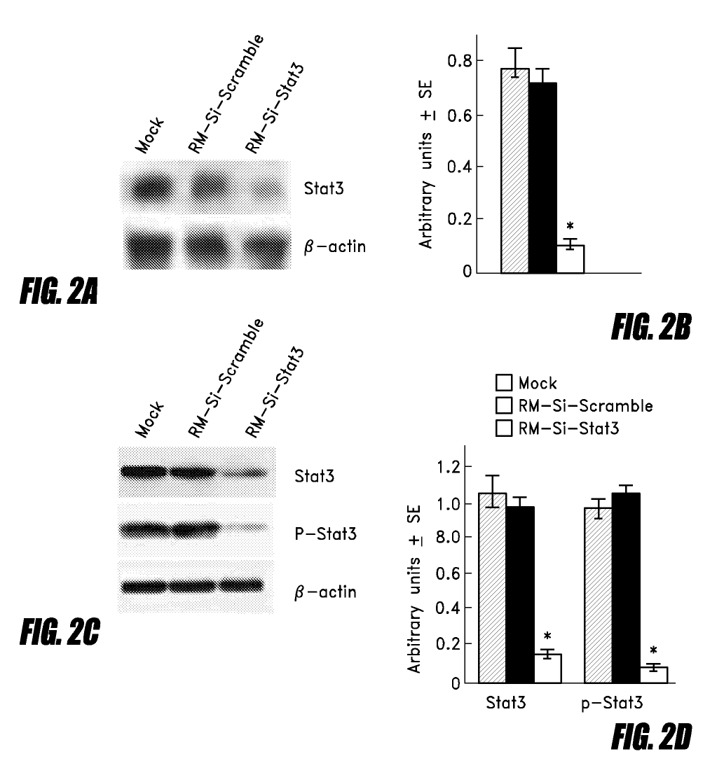

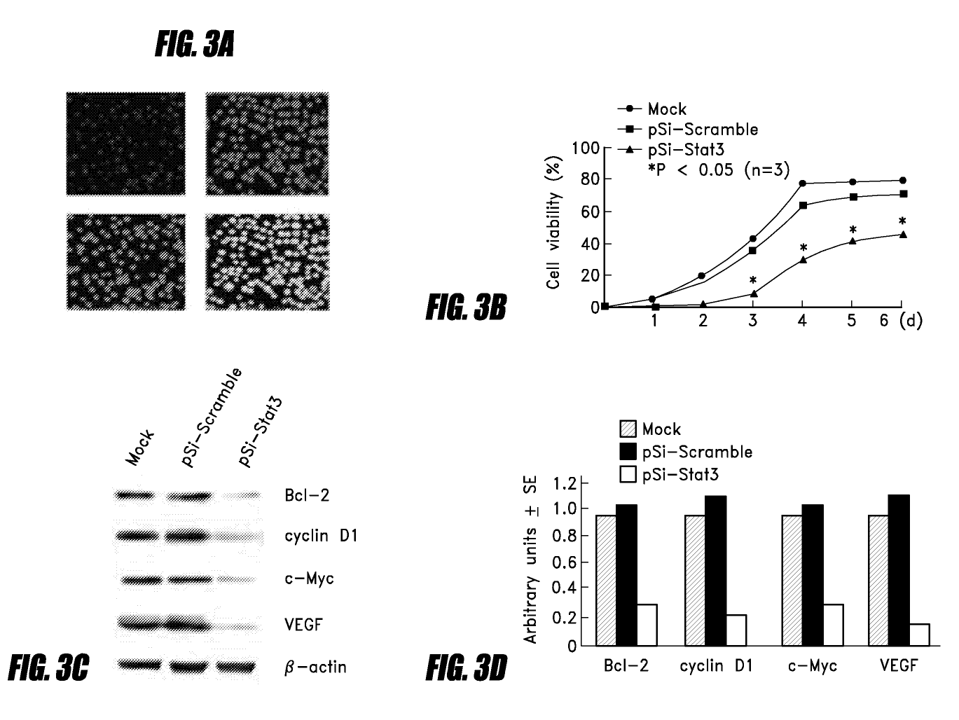

The ability of these constructs to silence Stat3 was determined next using Western and Northern blot analyses. The Stat3 mRNA level in RM-Si-Stat3 was reduced to ˜13% of that observed in RM-Si-Scramble (FIGS. 2A and B). Western blot analyses with native Stat3 (Stat3)- and phosphorylated Tyr⁷⁰⁵ Stat3 (p-Stat3)-specific antibodies also showed a strong inhibition of Stat3 or p-Stat3 proteins to ˜18% or 10%, respectively, in RMSi-Stat3 (FIGS. 2C and D) compared with RM-Si-Scramble. The percentages of STAT3 knockdown observed in Northern versus Western blot analyses are similar and statistically significant. Thus, the bacterially introduced Si-Stat3 specifically knocks down the expression of Stat3. We also examined the effects of siRNAs on cell growth and cycling. Cells were stained with acridine orange and subjected to flow cytometry. Stat3-siRNA induced significant apoptosis (˜23-fold) compared with the pSi-Scrambled control (Table 1). A further analysis of the flow cytometric data also showed that cells transfected with pSi-Stat3 accumulated significantly in G1 phase compared with the control (Table 1). These findings indicate that inhibition of Stat3 promotes both cessation of cell growth and enhancement of cell death. Because the Salmonella have been eliminated from the stable cell line by treatment with antibiotics, the effects on cell growth and cycling from the Si-Scramble control was equivalent to the uninfected mock group. Cells transfected with pSi-Stat3 grew slower and showed strong apoptosis (FIG. 3A) compared with those transfected with pSi-Scramble. Cells transfected with pSi-Stat3 became confluent 6 days after seeding, in contrast to the control group, which reached confluence by 4 days. In a separate experiment, cellular metabolic activity (as an indicator of cell viability) was measured using MTT assays in RM-1 cells transfected with the various plasmids. MTT data, expressed as tumor cell viability, were significantly decreased in the cells treated with the pSi-Stat3 compared with the control groups at day 6 (P<0.05, n=3; FIG. 3B). Stat3 has been shown to playa key role in promoting the cell cycle, proliferation, differentiation, and inhibition of apoptosis (Takeda, K. et al. 1999 Immunity 10:39-49). Persistently active Stat3 and its overexpression have been detected in a wide variety of human tumors (Catlett-Falcone, R. et al. 1999 Curr Opin Oncol 11:490-496), including prostate cancer (Mora, L. B. et al. 2002 Cancer Res 62:6659-6666). Constitutively active Stat3 promotes cell growth and survival via an overexpression of downstream targeted genes, such as the antiapoptotic Bcl-2, cell cycle regulators cyclin D1 and c-Myc, and inducers of tumor angiogenesis VEGF and MMP-2 (Musuda, M. et al. 2002 Cancer Res 62:3351-3355; Bromberg, J. F. et al. 1999 Cell 98:295-303; Alas, S, and Bonavida, B. 2001 Cancer Res 61:5137-5144; Puthier, D. et al. 1999 Eur J Immunol 29:3945-3950; Aoki, Y. et al. 2003 Blood 101:1535-1542; Niu, G. et al. 2002 Oncogene 2:2000-2008). We, therefore, examined if the expression of these genes was altered by Si-Stat3. The expression of Bcl-2, cyclin D1, c-Myc, VEGF, and MMP-2 was significantly knocked down in the presence of Si-Stat3 but not Si-Scramble (FIGS. 3C and D). Thus, the Stat3-specific shRNA interferes with the expression of tumor growth-promoting factors and decreases tumor cell survival.

### Tumorigenic Properties of RM-Si-Stat3 Cells In Vivo

We next examined the tumorigenic properties of RM-Si-Stat3 cells in vivo. C57BL6 mice (n=10) were injected with 2×10⁶ cells via the s.c. route into the upper flank, and tumor growth was monitored for 60 days. Mice transplanted with RM-Si-Scramble cells developed tumors at the injection sites by 21±3.6 days. In contrast, no tumors formed in the group injected with RM-Si-Stat3. Thus, the blockade of Stat3 reverses tumorigenicity of RM-1 prostate cancer cells.

Inhibition of Prostate Tumor Growth and Metastasis In Vivo by Bacterially Delivered shRNAs

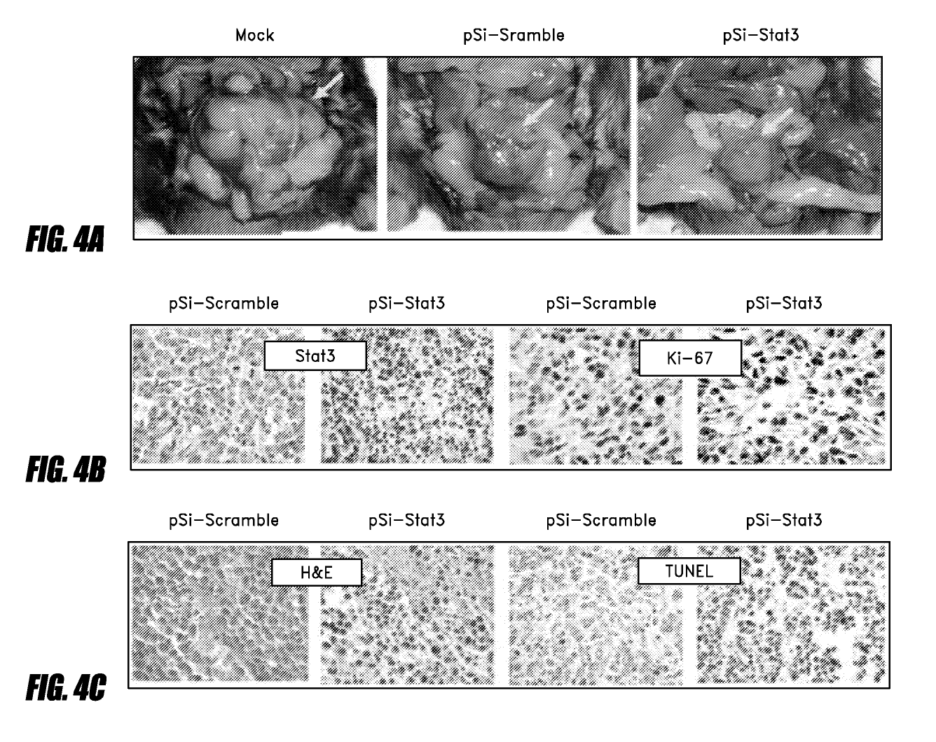

Although Salmonellae have been effective in retarding the growth of established tumors, complete tumor regression has never been proven. We, therefore, first studied the effects of S. typhimurium alone or combined with Stat3-specific siRNA in terms of prostate tumor growth and metastasis. To this end, we employed a C57BL6 mouse tumor implant model. A primary tumor was first established with RM-1 prostate carcinoma cells. Upon development of palpable s.c. tumors at the sites of inoculation, the tumor was excised and used for initiating primary prostate tumor development via an orthotopic surgical implantation of tumor tissues into recipient naive mouse prostates. Five days after tumor implantation, mice were divided into four groups (n=10 per group) and then injected with 1×10⁷ cfu of attenuated Salmonella carrying different plasmids via the tail vein. Eighteen days after bacterial injection, mice were sacrificed, and the tumors were excised, weighed, and measured. As shown in Table 2, mice treated with buffer alone (mock control) developed primary tumors with a mean volume of 2,458.51±602.18 mm³. In mice treated with Salmonella-Si-scramble, tumors grew to a volume of 589.22±380.34 mm³. In mice treated with Salmonella without any plasmid, the tumor grew to a comparable volume of 585.44±220.21 mm³. Thus, the bacteria carrying the scrambled-siRNA did not significantly affect tumor growth any differently compared with the Salmonella vector alone. However, mice treated with Salmonella-Si-Stat3 developed tumors with a median reduced volume of 216.42±134.15 mm³. Remarkably, tumors completely disappeared in one third of mice in this group over 18 days. The differences in tumor size between buffer control versus Salmonella-Si-scramble (P<0.05) and buffer control versus the Salmonella-Si-Stat3 group (P<0.01) were statistically very significant. The differences between Salmonella-Si-scramble or Salmonella alone versus Salmonella-Si-Stat3 group were also statistically significant (P<0.05). In summary, ˜3.9-fold higher tumor suppressive effect can be achieved with a single dose of bacteria transformed with a siRNA expression vector than those treated with Salmonella alone or Salmonella carrying Si-Scramble control, and ˜11.4-fold higher than those treated with buffer control (FIG. 4A, white arrowhead; Table 2). Thus, attenuated Salmonella alone exert an antitumor effect, which can be further enhanced by genetically modifying these organisms in combination with Stat3-specific siRNA expression.

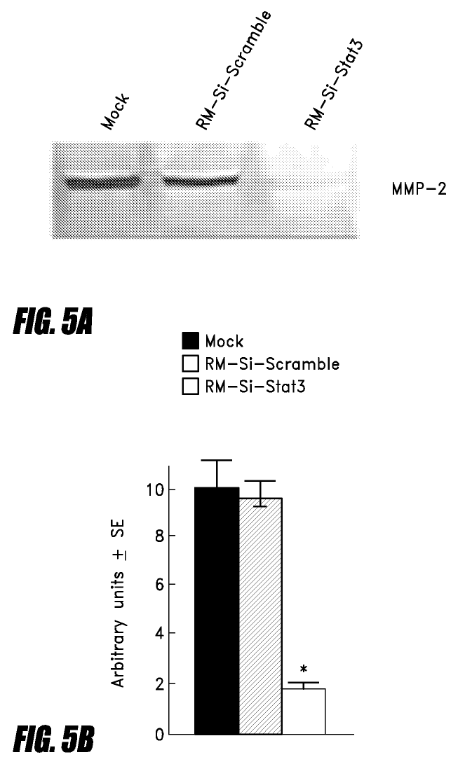

In addition to the primary tumor, metastases into liver, lung, spleen, kidney, and lymph nodes were examined in the recipient mice. A robust 84% reduction (P<0.01) in the numbers of metastases in the Salmonella-Si-Stat3-treated mice (Table 3) was observed. Tumor metastases occur primarily through tumor angiogenesis, aggressive growth of the primary tumor, and an extravasation of the tumor cells (Klein, C. A. 2004 Cell cycle 3:29-31). Secretion of extracellular proteases by the tumor plays an important role in metastasis (Klein, C. A. 2004 Cell cycle 3:29-31; Xie, T. X. et al. 2004 Oncogene 23:3550-3560). Among these, MMP-2/gelatinase A is believed to be essential for malignant behavior of cancer cells, such as rapid growth, tissue invasion, and metastasis (Klein, C. A. 2004 Cell cycle 3:29-31; Xie, T. X. et al. 2004 Oncogene 23:3550-3560). Consistent with this observation, we found that the MMP-2 activity in RM-1 cells significantly decreased after treatment with pSi-Stat3 compared with mock or pSi-Scramble (P<0.05; FIG. 5). Furthermore, blockade of Stat3 correlated with a reduction of expression of the Ki-67 protein, a proliferation-associated antigen (Yu, C. C. and Filipe, M. I. 1993 Histochem J 25:843-853). Immunohistochemical analyses for Stat3 and Ki-67 expression in the RM-1 tumor cells after transfection with Si-Scramble and in untreated RM-1 tumor cells were highly positive for Stat3 and Ki-67. In contrast, RM-1 tumor cells treated with pSi-Stat3 stained weakly for Stat3 and Ki-67 (FIG. 4B).

Tumors from mice treated with pSi-Scramble or pSi-Stat3 were excised for H&E staining and analyzed with TUNEL assays (FIG. 4C). pSi-Stat3-treated tumors show massive apoptosis with sparsely dispersed chromatin, several TUNEL-positive cells, and some necrotic regions compared with the Si-scramble control, which showed a finely granular cytoplasm with evenly dispersed chromatin and no TUNEL-positive cells. These data show that the Stat3 siRNA carried by Salmonella exerts a strong apoptotic antitumor effect in vivo.

The Attenuated S. typhimurium Expressing a Stat3-Specific siRNA Exerts a Robust Antitumor Effect

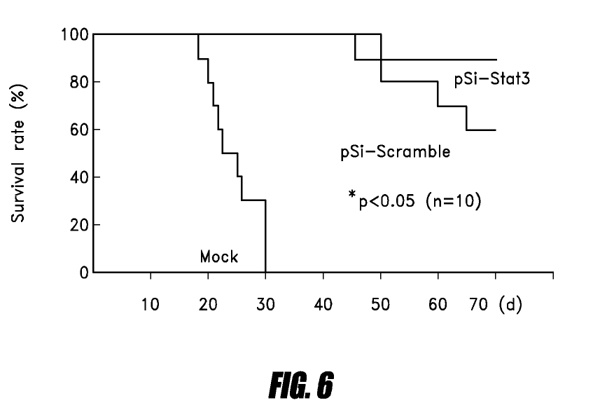

To further show the therapeutic utility of Salmonella-delivered siRNAs, tumor-bearing mice were injected with Salmonella carrying various plasmids or buffer. Mice were observed for 70 days. As shown in FIG. 6, all mice (n=10) injected with buffer were dead before 30 days. In contrast, the mice injected with Salmonella-Si-Stat3 and Salmonella-Si-Scramble had nine and six surviving mice at 70 days, respectively. These data clearly show that the attenuated Salmonella expressing a Stat3-specific siRNA exerts a robust antitumor effect.

### Recombinant Bacterial Distribution in C57BL6 Tumor-Bearing Mice

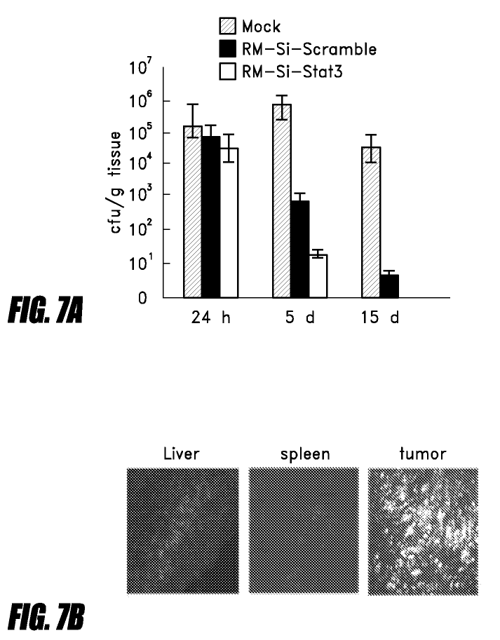

To determine if the potent antitumor effects of Salmonella with shRNA vectors was due to a preferential homing of bacteria into tumor tissue, we monitored the kinetics of bacterial distribution in C57BL6 tumor-bearing mice at specified times after injection of bacteria (FIG. 7A). Twenty-four hours after injection, similar numbers of bacteria were found in the liver, spleen, and tumors in tumor-bearing mice. The bacterial count (cfu) increased in tumors and decreased in the liver and spleen within 48 h after administration. By day 5, the number of bacteria in tumors increased significantly; the tumor to liver or tumor to spleen cfu ratio was 1,000:1 and 5,000:1, respectively, on average. By day 15, far more bacteria could be seen in the tumor compared with the liver, and no bacteria could be found in spleen tissues. On day 10, by using GFP expression as a marker, the bacterial distribution was also observed as markedly high in tumor tissue sections compared with those in spleen and liver tissue sections (FIG. 7B). At present, it is not clear why or how Salmonella specifically home to the tumor. Both characteristics of Salmonella and the heterogeneous microenvironments in solid tumors may combine to allow these bacteria to deliver therapeutic molecules preferentially to tumors. These characteristics may include (a) bacterial motility leading to uniform penetration within tumors; (b) hypoxic regions, an environment to which facultative anaerobic salmonellae are well adapted and can multiply, and in which macrophages, neutrophils, and granulocytes, effectors of bacterial clearance, are reduced in number (Chen, J. J. et al. 1998 Science 282:1714-1717); (c) both antibodies and serum complement components, which together can be lytic to salmonellae, are greatly restricted from the tumor environment by the irregular vasculature and positive pressure that exist inside tumors (Jain, R. K. 1991 Int J Radiat Biol 60: 85-100); (d) nutrients, such as high availability of glucose in aggressively growing tumors, may promote locally increased bacterial growth (Merida, I. and Avila-Flores, A. 2006 Clin Transl Oncol 8:711-716); and (e) Salmonella may induce apoptosis in infected macrophages (Monack, D. M. et 1996 Proc Natl Acad Sci USA 93: 9833-9838) at the tumor margins leading to increased antitumor inflammatory responses. An important recent advance in this field is the development of live, attenuated Salmonella vectors for DNA vaccine delivery (Shata, M. T. et al. 2001 Mol Med Today 6:66-71). The mechanisms involved in Salmonella delivery of DNA vaccine plasmids to the cytosol of mammalian cells is yet unclear (Alpuche-Aranda, C. M. et al. 1995 Infect Immun 63:4456-4462). However, several lines of evidence suggest that this bacterium can deliver nucleic acid vaccines in vivo, which elicit impressive levels of specific antibody response, T-cell proliferation, and CTL responses (Zoller, M. and Christ, O. 2001 J Immunol 166:3440-3450). Lastly, live Salmonella infection, but not Escherichia coli, induces the expression GRIM-19 (Bamich, N. et al. 2005 J Biol Chem 280:19021-19026), a protein inhibitor of STAT3 (Lufei, C. et al. 2003 EMBO J 22:1325-1335; Zhang, J. et al. 2003 Proc Natl Acad Sci USA 100:9342-9347). Thus, the potent antitumor effect of Salmonella can, in part, be due to an inhibition of STAT3 activity by increased GRIM-19 in the tumor. When these bacteria are combined with Stat3-specific siRNAs, a double-edged inhibitory effect may be exerted on STAT3 in vivo.

Our results provide the first convincing evidence that Salmonella can be used for delivering plasmid-based siRNAs into tumors growing in vivo. The Stat3-siRNAs carried by an attenuated S. typhimurium exhibit tumor suppressive effects not only on the growth of the primary tumor but also on the development of metastases, indicating that an appropriate attenuated S. typhimurium combined with the RNAi approach serves as the basis of a clinically feasible approach for cancer therapy. Ultimately, a live, attenuated Salmonella parenteral delivery system would likely be endotoxic in humans unless an msbB mutation was introduced, as reported previously (Low, K. B. et al. 1999 Nat Biotechnol 17:37-41).

### Salmonella Nomenclature

### Introduction

Salmonellosis is a major cause of bacterial enteric illness in both humans and animals. Each year an estimated 1.4 million cases of salmonellosis occur among humans in the United States. In approximately 35,000 of these cases, Salmonella isolates are serotyped by public health laboratories and the results are electronically transmitted to the Centers for Disease Control and Prevention (CDC). This information is used by local and state health departments and CDC to monitor local, regional, and national trends in human salmonellosis and to identify possible outbreaks of salmonellosis. Over the past 25 years, the National Salmonella Surveillance System has provided valuable information on the incidence of human salmonellosis in the United States and trends in specific serotypes. The recently implemented Salmonella Outbreak Detection Algorithm, another valuable tool for the recognition of outbreaks, allows users to detect increases in human infections due to specific Salmonella serotypes. Salmonella surveillance activities depend upon the accuracy of serotype identification and are facilitated by standardized nomenclature. The National Salmonella Reference Laboratory at CDC assists public health laboratories in the United States in serotype identification by providing procedure manuals, training workshops, updates, and assistance with the identification of problem isolates.

There are currently 2,463 serotypes (serovars) of Salmonella. The antigenic formulae of Salmonella serotypes are defined and maintained by the World Health Organization (WHO) Collaborating Centre for Reference and Research on Salmonella at the Pasteur Institute, Paris, France (WHO Collaborating Centre), and new serotypes are listed in annual updates of the Kauffinann-White scheme.

Salmonella nomenclature is complex, and scientists use different systems to refer to and communicate about this genus. However, uniformity in Salmonella nomenclature is necessary for communication between scientists, health officials, and the public. Unfortunately, current usage often combines several nomenclatural systems that inconsistently divide the genus into species, subspecies, subgenera, groups, subgroups, and serotypes (serovars), and this causes confusion. CDC receives many inquiries concerning the appropriate Salmonella nomenclature for the reporting of results and for use in scientific publications.

The nomenclature for the genus Salmonella has evolved from the initial one serotype-one species concept proposed on the basis of the serologic identification of O (somatic) and H (flagellar) antigens. Each serotype was considered a separate species (for example, S. paratyphi A, S. newport, and S. enteritidis); this concept, if used today, would result in 2,463 species of Salmonella. Other taxonomic proposals have been based on the clinical role of a strain, on the biochemical characteristics that divide the serotypes into subgenera, and ultimately, on genomic relatedness. The proposals for nomenclature changes in the genus have been summarized previously in the scientific literature.

The defining development in Salmonella taxonomy occurred in 1973 when investigators demonstrated by DNA-DNA hybridization that all serotypes and subgenera I, II, and IV of Salmonella and all serotypes of “Arizona” were related at the species level; thus, they belonged in a single species. The single exception, subsequently described, is S. bongori, previously known as subspecies V, which by DNA-DNA hybridization is a distinct species. Since S. choleraesuis appeared on the Approved List of Bacterial Names as the type species of Salmonella, it had priority as the species name. The name “choleraesuis,” however, refers to both a species and a serotype, which causes confusion. In addition, the serotype Choleraesuis is not representative of the majority of serotypes because it is biochemically distinct, being arabinose and trehalose negative.

In 1986 the Subcommittee of Enterobacteriaceae of the International Committee on Systematic Bacteriology at the XIV International Congress of Microbiology unanimously recommended that the type species for Salmonella be changed to S. enterica, a name coined in 1952, because no serotype shares this name. In 1987, investigators at the WHO Collaborating Centre formally made a proposal as a “Request for an Opinion” to the Judicial Commission of the International Committee of Systematic Bacteriology. The recommendation was adopted by CDC, in 1986 in the 4th edition of Edward's and Ewing's Identification of Enterobactericeae, and by other laboratories.

Nonetheless, the request was denied by the Judicial Commission. Although the Judicial Commission was generally in favor of S. enterica as the type species of Salmonella, its members believed that the status of Salmonella serotype Typhi, the causative agent of typhoid fever, was not adequately addressed in this request for an opinion. They were concerned that if S. enterica were adopted as the type species, Salmonella serotype Typhi would be referred to as S. enterica subsp. enterica serotype Typhi and might be missed or overlooked by physicians in the same way that S. choleraesuis subsp. choleraesuis serotype Typhi might be overlooked. From this perspective, nothing would be gained by changing the type species name. The Judicial Commission therefore ruled that S. choleraesuis be retained as the legitimate type species pending an amended request for an opinion. To comply with this ruling, an amended request was made, which is pending, to adopt S. enterica as the type species of Salmonella while retaining the species “S. typhi” as an exception.

In 1987, investigators also proposed that the seven subgenera of Salmonella be referred to as subspecies (subspecies I, II, IIIa, IIIb, IV, V, and VI). Subgenus III was divided into IIIa and IIIb by genomic relatedness and biochemical reactions. Subspecies IIIa (S. enterica subsp. arizonae) includes the monophasic “Arizona” serotypes and subspecies IIIb (S. enterica subsp. diarizonae) contains the diphasic serotypes. All “Arizona” serotypes had been incorporated into the Kauffmann-White scheme in 1979.

### The Current System Used by CDC

In Brenner, F. W. et al. 2000 J Clin Microbiol 38:2465-2467, investigators updated the nomenclature used at CDC for members of the genus Salmonella. The nomenclatural system is based on recommendations from the WHO Collaborating Centre and is summarized in Tables 4, 5, and 6.

According to the CDC system, the genus Salmonella contains two species, each of which contains multiple serotypes (Table 4). The two species are S. enterica, the type species, and S. bongori, which was formerly subspecies V. S. enterica is divided into six subspecies, which are referred to by a Roman numeral and a name (I, S. enterica subsp. enterica; II, S. enterica subsp. salamae; IIIa, S. enterica subsp. arizonae; IIIb, S. enterica subsp. diarizonae; IV, S. enterica subsp. houtenae; and VI, S. enterica subsp. indica). S. enterica subspecies are differentiated biochemically and by genomic relatedness.

CDC uses names for serotypes in subspecies I (for example, serotypes Enteritidis, Typhimurium, Typhi, and Choleraesuis) and uses antigenic formulas for unnamed serotypes described after 1966 in subspecies II, IV, and VI and in S. bongori (see discussion below). The name usually refers to the geographic location where the serotype was first isolated. For named serotypes, to emphasize that they are not separate species, the serotype name is not italicized and the first letter is capitalized (Table 5). At the first citation of a serotype the genus name is given followed by the word “serotype” or the abbreviation “ser.” and then the serotype name (for example, Salmonella serotype or ser. Typhimurium). Subsequently, the name may be written with the genus followed directly by the serotype name (for example, Salmonella Typhimurium or S. Typhimurium). CDC uses the format for formula designations used by the WHO Collaborating Centre. Both versions of the serotype name are listed as key words in manuscripts to facilitate the search and retrieval of information on Salmonella serotypes from electronic databases. Table 6 lists other serotype designations seen in the literature.

Serotype names designated by antigenic formulae include the following: (i) subspecies designation (subspecies I through VI), (ii) O (somatic) antigens followed by a colon, (iii) H (flagellar) antigens (phase 1) followed by a colon, and (iv) H antigens (phase 2, if present) (for example, Salmonella serotype IV 45:g,z₅₁:-. For formulae of serotypes in S. bongori, V is still used for uniformity (for example, S. V 61:z₃₅:-).

Before 1966 all serotypes in all subspecies except subspecies IIIa and IIIb were given names. In 1966 the WHO Collaborating Centre began naming serotypes only in subspecies I and dropped all existing serotype names in subspecies II, IV, and VI and S. bongori from the Kauffmann-White scheme. For surveillance purposes, i.e., for compatibility with old data, as stated above, CDC continues to use pre-1966 names for serotypes in subspecies II, IV, and VI and S. bongori. A common example of an old serotype name used at CDC and seen in the United States is S. ser. Marina (S. IV 48:g,z₅₁:-).

The majority (59%) of the 2,463 Salmonella serotypes belong to S. enterica subsp. I (S. enterica subsp. enterica) (19). Within S. enterica subsp. I, the most common O-antigen serogroups are A, B, C1, C2, D and E. Strains in these serogroups cause approximately 99% of Salmonella infections in humans and warm-blooded animals. Serotypes in S. enterica subspecies II (S. enterica subsp. salamae), IIIa (S. enterica subsp. arizonae), IIIb (S. enterica subsp. diarizonae), IV (S. enterica subsp. houtenae), IV (S. enterica subsp. indica), and S. bongori are usually isolated from cold-blooded animals and the environment but rarely from humans.

### Conclusions

The nomenclature for Salmonella is still evolving and the debate on the name for the type species is not likely to be settled any time soon. In the meantime, the work of isolating, identifying, and reporting on Salmonella serotypes must go on for diagnostic, therapeutic, and public health purposes. The nomenclature system used at CDC, essentially based on the recommendations established by the WHO Collaborating Centre, is believed to adequately address the concerns and requirements of clinical and public health microbiologists. Because the type species name has not been officially approved and in order to shorten reports, Salmonella enterica subsp. enterica serotype Typhimurium, for example, is shortened to Salmonella serotype (ser.) Typhimurium or Salmonella Typhimurium. To ensure backward compatibility with literature searches on Salmonella serotypes from electronic databases, both versions of the serotype name should be listed as key words in manuscripts. In 1999, at the American Society for Microbiology (ASM) Publications Board Meeting, a proposal that relevant ASM journals adopt the Salmonella nomenclature currently used at CDC was unanimously endorsed by the board, with plans to update 2000 ASM Instructions to the Authors.

Recent Advances in the Development of Live Attenuated Salmonella Vectors

Much of the progress in developing bacterial vectors that are suitable for use in humans has been made through the evolution of mutant strains of pathogenic bacteria such as Salmonella enterica (serovars Typhi and Typhimurium), Listeria monocytogenes, Shigella flexneri and Vibrio cholerae (Roland, K. L. et al. 2005 Curr Opin Molec Ther 7:62-72). The tissue tropism of these organisms naturally targets inductive sites of the host immune system, such as mucosal surfaces and antigen-presenting cells, and influences T-lymphocyte responses to both homologous and heterologous antigens. Many vectors under development such as Listeria and Shigella are facultative, intracellular bacteria that replicate within host cells as well as extracellularly. The release of an internalized bacterial vector from phagocytic vacuoles into the host cell cytoplasm is an effective method for delivering macromolecules such as protein antigens and plasmid DNA. Cytoplasmic processing of the internalized vector and passenger antigen and immunological presentation by major histocompatibility complex class I molecules stimulates the enhanced production of CD8+ T-lymphocytes. Pathogenic Salmonellae are also capable of replicating in the host cell cytosol but remain internalized in phagocytic vacuoles. Immunization with attenuated Salmonella typhi primes the host to elicit both humoral and cellular immune systems, enhanced by the induction of mucosal immune responses. The tropism of Salmonella for solid tumors is currently being exploited to deliver anticancer agents and is augmented by the presence of lipopolysaccharide (LPS) and other cell-associated components that trigger the release of cytokines and pro-inflammatory mediators, such as tumor necrosis factor (TNF)α and nitric oxide. Finally, organisms such as V. cholerae, which are non-invasive elicit the production of strong systemic (serum immunoglobulin, IgG) and mucosal (secretory IgA) antibody responses and provide a new strategy for vaccines that prevent infection at mucosal surfaces.

### Live, Attenuated Salmonella Vaccines and Vectors

### Salmonella Vaccines

Attenuated mutants of Salmonella enterica serovar Typhi (S. typhi) and Typhimurium (S. typhimurium) have been studied extensively in both preclinical and clinical trials as multivalent vectors expressing a variety of different bacterial, viral and protozoal antigens. Much of the early interest in using attenuated Salmonella vectors was predicated on extensive studies of S. typhi Ty21a, the only live bacterial vaccine licensed for use against typhoid fever. However, Ty21a is weakly immunogenic and genetically undefined, making it inefficient as a vector. Orally administered, single-dose, typhoid fever vaccine candidates, including S. typhi CVD-908-htrA (ΔaroACΔhtrA; HolaVAx-Typhoid vaccine, Berna Biotech AG), Ty800 (ΔphoPQ; AVANT Immunotherapeutics Inc.), and ZH9 (ΔaroCΔssaV; typhoid vaccine, Microscience Ltd.) were subsequently created by the deletion of known genes from virulent strains of S. typhi. Phase I and II clinical studies with these typhoid fever vaccine candidates demonstrated that they were well tolerated and immunogenic.

Development of Attenuated, S. typhi Vectors

The potential application of newly developed vaccine strains as vectors for delivering one or more heterologous antigen(s) was recently demonstrated in clinical studies conducted by Microscience Ltd (Table 7). In a Phase I safety and immunogenicity study, 36 volunteers were administered recombinant S. typhi ZH9 expressing the heat-labile toxin (LT) B-subunit (LT-B) of Escherichia coli (ZH9/LT-B). An aroCΔssaV S. typhimurium mutant expressing LT-B was previously highly immunogenic in mice, eliciting the production of high-titer, anti-LT serum antibody, as well as modest levels of LT-specific mucosal IgA. In clinical trials, ZH9/LT-B was well tolerated and immunogenic, as 70% of vaccinees seroconverted to the vaccine antigen following two oral doses. This vaccine has potential applications for both travelers and military personnel deployed to areas endemic for enterotoxigenic E. coli (ETEC). More recently, a small, 30-patient phase I study showed that two orally administered doses of ZH9 expressing the hepatitis B virus; core antigen (HBcAg) elicited the production of proliferative, T-lymphocyte responses to HBcAg in all vaccinees. No significant adverse events were reported. The results from these studies clearly highlight the potential of new, typhi-based vectors for prophylactic and therapeutic use.

### Salmonella Vectors and Bioterrorism/Warfare

Live, attenuated Salmonella vectors have conferred rapid protection in experimental challenge models to biological agents transmitted via the mucosa. These results have stimulated a renewed interest in developing new vector vaccines to address the increased threat of bioterrorism and warfare. Early preclinical studies showed that an orally administered Salmonella aroA mutant expressing anthrax toxin protective antigen (PA) in the bacterial periplasm conferred partial protection to spore challenge in mice, however, PA-specific antibody responses were not detected in the sera of immunized animals. In addition, the PA transgene was expressed from a multicopy plasmid that was unstable due to both high copy number and the need to stabilize it with antibiotics. These results were recently expanded using an aroA Salmonella mutant engineered to secrete PA into the extracellular environment with the signal sequence of hemolysin (Hly) A from E coli. A single gene copy encoding a translational fusion of PA to HlyA, and the necessary accessory genes, were integrated into the Salmonella chromosome in an arrangement intended to stabilize PA expression in the absence of antibiotics. Mice immunized intravenously with three doses of this recombinant Salmonella mutant developed high-titer serum anti-PA antibody that protected against intraperitoneal challenge with virulent anthrax bacteria. In contrast, mice immunized with three oral doses of the same strain elicited the production of only low levels of PA-specific antibody. The extent of secretory IgA production or another indicator of a mucosal immune response in orally immunized mice was not reported. More recently, an anthrax vaccine candidate based on the typhoid fever vaccine strain S. typhi Ty21a was described. This recombinant strain expressed intracellular PA via an inducible promoter when examined in culture under conditions of oxygen limitation. Preliminary studies in mice demonstrated PA-specific immune stimulation following a single, parenteral injection.

Attenuated Typhi recombinants have also been developed recently as potential vaccines against plague. Early studies showed that expression of the Yersinia pestis F1 capsule antigen in attenuated S. typhimurium elicited protective immunity in mice against virulent, encapsulated plague bacteria. Recently, an attenuated mutant of S. typhi (ΔaroACΔhtrA) was created to express the F1 antigen on its surface through the introduction of the Y. pestis capsule operon (caf) on a plasmid with low copy number. Intranasal immunization of mice with this recombinant S. typhi strain elicited the production of both serum IgG and mucosal IgA antibodies specific for F1. Subcutaneous challenge of these mice with Y. pestis revealed that although mice were protected, there was no clear correlation between protection and levels of F1-specific serum IgG. Cellular assays to determine if a cellular component of immunity was involved were not performed. To be effective, subsequent vaccine candidates will likely incorporate Y. pestis V antigen in order to provide additional protection against unencapsulated strains of Y. pestis.

Development of Attenuated S. typhimurium Vectors

While attenuated S. typhimurium vectors have been extensively characterized in animals, it was only recently that investigators turned their attention to the use of non-typhoidal Salmonella-based vectors in humans. The development of Typhimurium vectors is based on the notion that the prolonged intestinal phase of the organism may induce an immune response in the gastrointestinal system that is qualitatively and/or quantitatively different than that elicited by S. typhi. This concept was originally tested using S. typhimurium LH1160, a strain bearing mutations in the phoPQ and purB genes and complemented by a PurB-expressing balanced-lethal plasmid encoding the Helicobacter pylori ureAB genes. Balanced-lethal plasmid expression systems were first developed for use in attenuated strains of Salmonella to address issues of plasmid instability and the requirement for antibiotic resistance markers. The vaccine was generally well tolerated by volunteers who received a single, oral dose and there were no reports of bacteremia or serious diarrhea, although a few individuals developed low-grade fever. Most of the vaccinees developed specific mucosal and humoral anti-Salmonella immune responses. More significantly, half of the volunteers also developed antibody responses to UreAB. Based on the modest reactogenicity of LH1160, investigators attenuated this strain further by introducing a deletion in the aroA gene. A secretion system was then engineered to express a fusion protein of HIV-1 Gag to the amino terminus of SopE, a component of the S. typhimurium type III secretion system. The fusion gene was expressed from the native sopE promoter and maintained in the bacterial vector on a stable, low-copy, Asd-based balanced-lethal plasmid. In a recent study conducted at the New England Regional Primate Center, similar ΔphoPQ-deleted strains of S. typhimurium and S. typhi were used as vectors to deliver fragments of the simian immunodeficiency virus (SIV) Gag protein fused to SopE. Transient, low level cytotoxic T-lymphocyte (CTL) responses to a SIV Gag epitope were detected in Rhesus macaques, following each dose of Salmonella. This study demonstrated the potential of mucosal priming by the Salmonella type III secretion system to direct SIV-specific cellular immune responses to the gastrointestinal mucosa in a primate model. A phase I dose-escalation human trial is currently being conducted at the Massachusetts General Hospital to evaluate the safety and immunogenicity of an attenuated S. typhimurium ΔphoPQ ΔaroA strain expressing SopE fused to an HIV-I Gag epitope. Thus far, volunteers have tolerated vaccine doses as high as 5×10⁸ colony forming units (CFU) without significant side effects.

Using similar technology, a single-dose, orally administered plague vaccine is currently being developed by AVANT Immunotherapeutics Inc. Strain M020 was attenuated through the deletion of the Salmonella phoPQ virulence regulon and bears an additional deletion of the asd gene. S. typhimurium M020 harbors a multifunctional plasmid that encodes Asd and a genetic fusion of the Y. pestis F1 and V antigens (F1-V). Strain M020 was genetically stable during laboratory growth and expressed moderate levels of F1-V that remained localized in the bacterial cytoplasm. M020 elicited the production of high-titer IgG antibodies to both Salmonella and F1-V in mice fed two doses of the vaccine. Higher immune responses against M020 were observed in a recently developed rabbit immunogenicity model. AVANT is currently preparing M020 for clinical studies.

### Salmonella Vectors and Cancer Treatment

Cancer therapeutics is a promising new area for Salmonella vector research. It has been recognized for some time that bacteria such as Salmonella, Clostridium and Bifidobacterium have a natural tropism for solid tumors, and that this tropism may be exploited to facilitate the selective delivery of therapeutic agents to tumor cells. Potential applications for this technology include vectors for gene therapy, delivery of therapeutic drugs such as interleukin (IL)-2, or prodrug-converting enzymes. Attenuated Salmonellae have also been recently employed as vectors to deliver eucaryotic expression plasmids to the secondary lymphoid tissues considered to be essential for eliciting the production of antitumor responses by DNA vaccines.

S. typhimurium VNP-20009 (Vion Pharmaceuticals Inc) was created by the chromosomal deletion of two genes, purl (purine biosynthesis) and msbB (LPS biosynthesis), and was attenuated by at least 10,000-fold in mice compared with the parental wild-type strain. The results from a phase I safety study showed that humans could safely tolerate high doses (10⁹ CFU/m²) of VNP-20009, while remaining colonized with the vector for up to 2 weeks. VNP-20009 was subsequently modified to express the prodrug-converting enzyme, cytosine deaminase (CD) CD converts 5-fluorocytosine (5-FC) to 5-fluorouracil (5-FU), a deaminated form of 5-FC that is highly cytotoxic for eucaryotic cells. Preclinical studies showed that tumor-bearing animals immunized with VNP-20009 had a >90% reduction in tumor growth in several murine tumor models, and that reduction corresponded to the presence of high levels of 5-FU in animals injected post-immunization with 5-FC.

Attenuated Salmonella vectors have been used to deliver plasmid DNA encoding tumor-specific antigens or T-cell epitopes to elicit the production of anticancer immunity. This approach poses a number of challenges, including the concept of overcoming peripheral T-cell tolerance to ‘self’ antigens. Support for this approach was first demonstrated using a tumor-associated model antigen (β-galactosidase) expressed by an S. typhimurium aroA mutant. Orally vaccinated mice produced both antigen-specific humoral (antibody) and cell-mediated immune responses (CTL), and showed long-term protection against a fibrosarcoma expressing β-galactosidase. During the last few years, a number of orally administered, Salmonella based gene delivery systems expressing recognized, tumor-associated antigens were evaluated in mice and elicited the production of protective immune responses against experimental forms of cancer. More recently, investigators have taken the novel approach of using Salmonella-based gene delivery systems to inhibit tumor growth and metastasis by attacking a tumor's blood supply. A promising example is an orally administered, aroA Salmonella mutant expressing vascular endothelial growth factor receptor-2 (Flk-1). Following three oral doses, this mutant elicited the production of CTLs in mice, which markedly inhibited the growth of subcutaneous tumors in a melanoma tumor model. More impressively, this same mutant elicited the production of prolonged antitumor effects in mice subjected to tumor challenge in a murine colon carcinoma model. These results are supported by more recent studies in which parenteral administration of an attenuated Salmonella choleraesuis mutant harboring eucaryotic expression vectors encoding the angiogenic inhibitors endostatin or thrombospondin-1 enhanced CD8+ T-lymphocyte infiltration and significantly decreased intratumoral microvascularization, prolonging, in some cases, survival of vaccinated mice. Gene delivery using attenuated Salmonella vectors to target tumor vasculature appears to hold particular promise for the treatment of primary and metastatic melanomas and other solid tumors.

Strategies for Silencing Human Disease Using RNA Interference

The realm of RNA interference (RNAi) has expanded at a remarkable rate since the initial characterization of RNAi in the nematode Caenorhabditis elegans. Soon after this, RNAi was shown to occur in mammalian cells in response to double-stranded small interfering RNAs (siRNAs) of ˜21 nt in length that serve as the effector molecules of sequence-specific gene silencing. Mechanistic insights followed rapidly during the ensuing years, and with them came the increasing hope that RNAi pathways could be harnessed for the therapeutic intervention of human diseases. The key therapeutic advantage of using RNAi lies in its ability to specifically and potently knock down the expression of disease-causing genes of known sequence. Furthermore, the relatively short turnaround time for efficacy testing of potential therapeutic RNAi molecules, and the fact that even newly discovered pathogens are theoretically amenable to rapid targeting, has caused great excitement about the potential of RNAi for treating a wide range of diseases (Kim, D. H. and Rossi, J. J. 2007 Nat Rev Genet. 8:173-184).

Recent findings have highlighted the effectiveness of RNAi in therapeutically relevant settings, the results of which have spurred cautious optimism about the promise of RNAi-based therapies. The first clinical applications of RNAi have been directed at the treatment of wet, age-related macular degeneration (AMD) and respiratory syncytial virus (RSV) infection. Therapies based on RNAi are also in preclinical development for other viral diseases, neurodegenerative disorders and cancers, although a number of challenges need to be addressed and improvements made for RNAi-based therapies to realize their full potential. A progressively more detailed understanding of the basic mechanisms of RNAi has been important in developing diverse RNAi effector molecules with improved levels of potency and efficacy. For example, synthetic siRNAs and expressed short hairpin RNAs (shRNAs) both have specific advantages and disadvantages, which are important considerations when designing RNAi-based therapies for a particular disease. In addition, although many in vivo studies have shown the potential effectiveness of various RNAi-based strategies, other studies have highlighted challenges that arise as a result of using an endogenous cellular mechanism for therapeutic benefit. Unwanted side effects have included induction of type 1 interferon (IFN) responses and saturation of endogenous RNAi pathway components, indicating that caution is necessary when designing effector molecules for delivery into target cells. The issue of cell-specific or tissue-specific delivery is another key challenge in developing RNAi-based therapies. Various strategies for non-viral and viral delivery of RNAi triggers have recently been shown to be effective in disease models, raising the hope that clinical studies of RNAi-based therapies will be extended to an increasing list of diseases in the near future.

### Mechanisms of RNAi-Mediated Gene Silencing

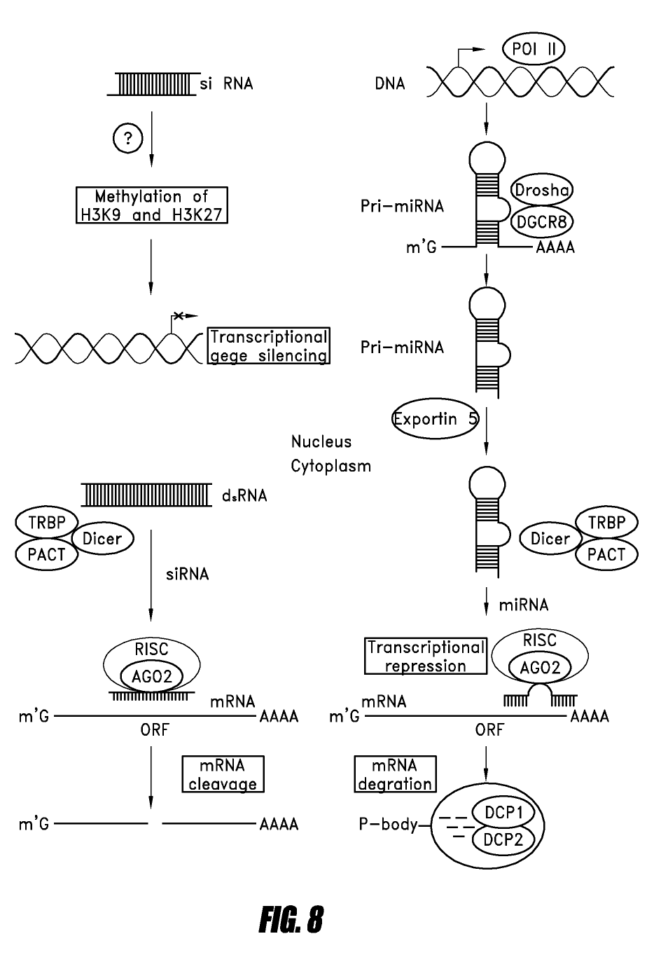

RNAi pathways are guided by small RNAs that include siRNAs and microRNAs (miRNAs), which derive from imperfectly paired hairpin RNA structures naturally encoded in the genome. RNAi effector molecules induce gene silencing in several ways: they direct sequence-specific cleavage of perfectly complementary mRNAs and translational repression and transcript degradation for imperfectly complementary targets. RNAi pathways can also direct transcriptional gene silencing (TGS) in the nucleus, although mechanistic details of TGS are not yet well established in mammalian systems (FIG. 8).

Referring to FIG. 8, as shown in the pathway at the bottom left, cytoplasmic double-stranded RNAs (dsRNAs) are processed by a complex consisting of Dicer, TAR RNA-binding protein (TRBP) and protein activator of protein kinase PKR (PACT) into small interfering RNAs (siRNAs), which are loaded into Argonaute 2 (AGO2) and the RNA-induced silencing complex (RISC). The siRNA guide strand recognizes target sites to direct mRNA cleavage, which is carried out by the catalytic domain of AGO2. siRNAs complementary to promoter regions direct transcriptional gene silencing in the nucleus through chromatin changes involving histone methylation (top left); the precise molecular details of this pathway in mammalian cells are currently unclear. As shown in the pathway on the right, endogenously encoded primary microRNA transcripts (pri-miRNAs) are transcribed by RNA polymerase II (Pol II) and initially processed by Drosha-DGCR8 (DiGeorge syndrome critical region gene 8) to generate precursor miRNAs (pre-miRNAs). These precursors are exported to the cytoplasm by exportin 5 and subsequently bind to the Dicer-TRBP-PACT complex, which processes the pre-miRNA for loading into AGO2 and RISC. The mature miRNA recognizes target sites in the 3′ untranslated region (3′ UTR) of mRNAs to direct translational inhibition and mRNA degradation in processing (P)-bodies that contain the decapping enzymes DCP1 and DCP2. H3K9, histone 3 lysine 9; H3K27, histone 3 lysine 27; m⁷G, 7-methylguanylate; ORF, open reading frame.

Post-Transcriptional Gene Silencing by siRNAs

Exogenous siRNAs target complementary mRNAs for transcript cleavage and degradation in a process known as post-transcriptional gene silencing (PTGS). In nematodes, insects and plants, this pathway functions as an innate antiviral defense mechanism, in which viral double-stranded RNA (dsRNA) molecules are processed by the RNase III enzyme Dicer into siRNAs that mediate the RNAi response. Whether or not siRNA-mediated PTGS exists in mammalian cells for intrinsic immunity against viral infections is unclear, and remains an area for further investigation.

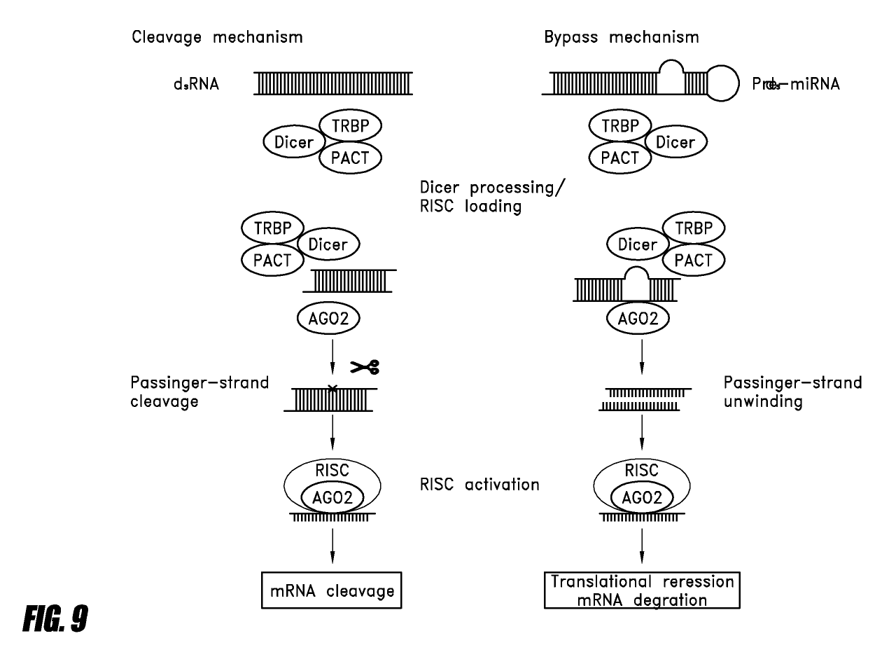

Effective PTGS requires perfect or near-perfect Watson-Crick base pairing between the mRNA transcript and the antisense or guide strand of the siRNA, and results in cleavage of the mRNA by the RNA-induced silencing complex (RISC). The endonuclease Argonaute 2 (AGO2) is responsible for the cleavage mechanism of RISC, and AGO2 is the only member of the Argonaute subfamily of proteins with observed catalytic activity in mammalian cells. RISC activation is initially thought to involve AGO2-mediated cleavage of the sense or passenger strand of the double-stranded siRNA, generating the single-stranded antisense strand that serves to guide RISC to complementary sequences in target mRNAs (FIG. 9). This guide strand is bound within the catalytic, RNase H-like PIWI domain of AGO2 at the 5′ end and a PIWI-Argonaute-Zwille (PAZ) domain that recognizes the siRNA 3′ end.

Referring to FIG. 9, double-stranded RNAs (dsRNAs) and precursor microRNAs (pre-miRNAs) are processed by a complex comprising Dicer, TAR RNA-binding protein (TRBP) and protein activator of protein kinase PKR (PACT), facilitating loading of the small interfering RNA (siRNA) or microRNA (miRNA) duplex into Argonaute 2 (AGO2) and RNA-induced silencing complex (RISC). When the RNA duplex loaded into RISC has perfect sequence complementarity, AGO2 cleaves the passenger strand so that active RISC is produced that contains the guide strand, which is complementary to the target sequence. When the RNA duplex loaded into RISC has imperfect sequence complementarity a bypass mechanism is used, in which a helicase activity is required to unwind the passenger strand from the guide strand and to generate the mature miRNA strand, producing active RISC.

The cleavage of targeted mRNA takes place between bases 10 and 11 relative to the 5′ end of the siRNA guide strand, leading to subsequent degradation of the cleaved mRNA transcript by cellular exonucleases. On activation by the siRNA guide strand, RISC can undergo multiple rounds of mRNA cleavage to mediate a robust PTGS response against the target gene. PTGS by mRNA cleavage has been exploited as the method of choice for potential therapeutic applications of RNAi because of the potency of this catalytic gene-silencing pathway.

The microRNA Pathway

The endogenous miRNA pathway serves as a cellular rheostat for fine-tuning gene expression during development and differentiation. The 3′ untranslated regions (3′ UTRs) of mRNAs are targeted by miRNAs with which they share partial sequence complementarity. These endogenous small RNAs of 22 nt in length induce PTGS through translational repression. This is often accompanied by subsequent mRNA degradation, which occurs in cytoplasmic compartments known as processing bodies (P-bodies). When an miRNA has complete sequence complementarity with a target mRNA, it instead directs cleavage of the mRNA transcript through RISC activity. One such example, miR-196-directed cleavage of Hoxb8 (homeobox 8), has been shown to occur in mammalian cells, illustrating a level of functional overlap between siRNA and miRNA-directed gene-silencing pathways.

Long primary miRNA transcripts (pri-miRNAs) are generally transcribed by RNA polymerase II (Pol II) in the nucleus (although a recent finding also describes miRNAs transcribed by RNA Pol III) and are processed by the RNase III enzyme Drosha into 70 nt stem-loop structures known as precursor miRNAs (pre-miRNAs). Drosha functions with the dsRNA-binding protein of DiGeorge syndrome critical region gene 8 (DGCR8) in a complex known as the microprocessor to generate these pre-miRNAs. The dsRNA-binding protein exportin 5 then transports the pre-miRNA into the cytoplasm in a Ran-GTP-dependent manner, where Dicer and its dsRNA-binding protein partners, HIV-1 TAR RNA-binding protein (TRBP) and protein activator of protein kinase PKR (PACT), process the pre-miRNA and load the 22 nt mature miRNA into RISC42 (FIG. 8). The miRNA-loading pathway into RISC does not seem to involve cleavage of the miRNA passenger strand, and might instead use a bypass mechanism that requires helicase activity to unwind and discard the passenger strand; imperfect sequence homology between the mature miRNA strand and its complementary passenger strand might prevent AGO2 from cleaving the passenger strand. Once the passenger strand has been unwound or discarded and the mature miRNA binds to its target mRNA 3′UTR, RISC directs translational repression and subsequent mRNA degradation to silence gene expression (FIG. 9). The seed sequence of a mature miRNA, which encompasses the first 2-7 or 2-8 nucleotides from its 5′ end, must have complete complementarity with its target, whereas mismatched nucleotides in the 3′ end of the miRNA strand are more tolerated. Although the effector stages of this endogenous pathway have not been used for therapeutic development, perfect duplex siRNA sequences have been introduced into pri-miRNA and pre-miRNA backbones, generating miRNA mimics that are processed by the miRNA pathway but trigger the more potent PTGS pathway of mRNA cleavage once loaded into RISC.

Transcriptional Gene Silencing by siRNAs

Silencing of gene expression at the transcriptional level was first shown to take place in the nuclei of plant and fungal cells. TGS regulates gene expression through changes in chromatin mediated by siRNAs and the RNAi machinery (FIG. 8). In mammalian cells, some level of TGS and histone methylation has been shown to occur in response to exogenous, promoter-targeting siRNAs, although the precise mechanism by which this is achieved is poorly understood. TGS might potentially be used in future therapeutic applications of RNAi for prolonged, epigenetic gene silencing, but no such applications have been tested in preclinical models so far.

### Designing Potent Triggers of RNAi

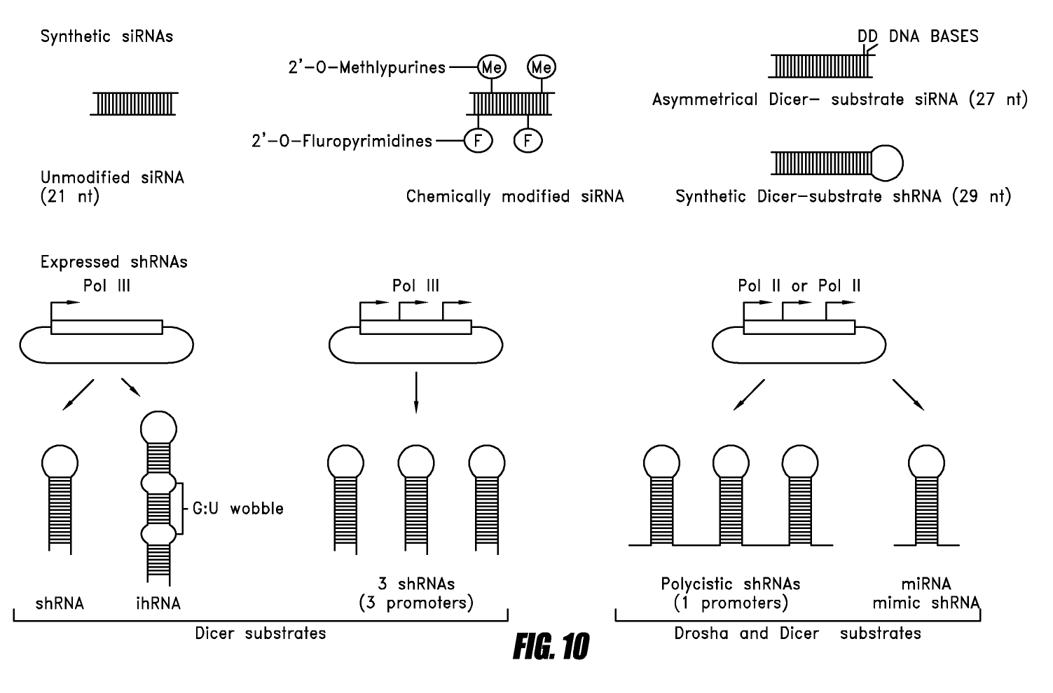

Delivered siRNAs: Most of the proposed clinical applications of RNAi incorporate chemically synthesized 21-nt siRNA duplexes that have 2-nt 3′ overhangs (FIG. 10a), allowing large-scale synthesis and uniform production of siRNA molecules that are also amenable to chemical modifications that increase their stability. Knockdown of gene expression is accomplished by designing siRNA sequences that target the coding and non-coding regions of mRNAs with perfect complementarity to induce PTGS. Several commercial entities involved in the manufacturing of siRNAs provide effective design algorithms online, which are based on a combination of mRNA target sequence and secondary structures, siRNA duplex end-stabilities, and aim to minimize potential sequence-dependent OTEs.

Referring to FIG. 10a, synthetic small interfering RNAs (siRNAs; left panel) are administered in vivo. These can be produced with chemical modifications (middle panel), such as 2′-O-methylpurines or 2′-fluoropyrimidines, which can be added to increase stability. Asymmetrical Dicer-substrate siRNAs can also be produced (right panel). These have a blunt end that includes two DNA bases (D), whereas the other end has a 2-nt 3′ overhang. This ensures that a single species of siRNA is generated by Dicer, which processes the blunt end. Longer synthetic short hairpin RNAs (shRNAs) are also processed as Dicer substrates. As shown in FIG. 10b, expression vectors drive high levels of shRNA expression from polymerase III (Pol III) promoters (left panel). Long hairpin RNAs (IhRNAs) generate multiple Dicer-processed siRNA species, suggesting mammalian Dicer is processive. Multiple separate Pol III promoters can be used in one vector to drive expression of several different shRNAs (middle panel). Vectors carrying Pol II or Pol III promoters generate longer precursor RNAs, including polycistronic shRNA transcripts and microRNA (miRNA) mimics that are processed by both Drosha and Dicer (right panel).

Longer siRNAs (27mers) and shRNAs (29 nt) that are chemically synthesized serve as substrates for Dicer processing (FIG. 10a), and for some siRNA-target combinations the use of these longer dsRNAs can increase the potency of PTGS. Dicer and TRBP-PACT might comprise a loading platform for RISC formation, and incorporating this loading step in the RNAi pathway through the use of Dicer substrates elicits a more potent gene-silencing effect. 27-mers are designed so that they are asymmetrical, with one 2-nt 3′ overhang and one blunt end. Because Dicer recognizes the 2-nt 3′ overhang for processing, this design ensures that a single siRNA product is produced. However, the blunt end, which includes DNA bases, might trigger low levels of interferon induction, but the lower concentrations of 27-mers required to silence gene expression might avoid or minimize such an interferon response.

Expressed shRNAs: siRNAs transiently silence gene expression, because their intracellular concentrations are diluted over the course of successive cell divisions. By contrast, expressed shRNAs mediate long-term, stable knockdown of their target transcripts for as long as transcription of the shRNAs takes place (FIG. 10b). RNA Pol II and III promoters are used to drive expression of shRNA constructs, depending on the type of expression required. Consistent with their normal cellular roles in producing abundant, endogenous small RNAs, Pol III promoters (such as U6 or H1) drive high levels of constitutive shRNA expression, and their transcription initiation points and termination signals (4-6 thymidines) are well defined. Pol II promoter-driven shRNAs can be expressed tissue-specifically and are transcribed as longer precursors that mimic pri-miRNAs and have cap and polyA signals that must be processed. Such artificial miRNAs/shRNAs are efficiently incorporated into RISC, contributing to a more potent inhibition of target-gene expression; this allows lower levels of shRNA expression and might prevent saturation of components in the RNAi pathway. An additional advantage of Pol II promoters is that a single transcript can simultaneously express several miRNA and mimic shRNAs (FIG. 10b). This multiplexing strategy can be used to simultaneously knock down the expression of two or more therapeutic targets, or to target several sites in a single gene product.

### Cancer

Oncogenes expressed at abnormally high levels are attractive targets for RNAi-based therapies against cancers, and such approaches have effectively inhibited tumor growth in vivo in mouse models. One successful study involved liposomal delivery of siRNAs targeting the tyrosine kinase receptor EphA2 gene, which is overexpressed in ovarian cancer cells. After biweekly delivery of siRNAs for 4 weeks, an up to 50% reduction of tumor size was observed. When RNAi therapy was combined with the chemotherapy agent paclitaxel, an up to 90% reduction in tumor size was observed, indicating the potency and effectiveness of combining RNAi with conventional forms of therapy, especially for cancers.

In another example, metastatic Ewing sarcoma cells have been successfully targeted in a mouse model using cyclodextrin nanoparticles to systemically deliver siRNAs targeting the Ews-FliI gene fusion. Tumor growth in vivo was suppressed after systemic delivery of siRNA-containing nanoparticles. Of greater significance, however, was that the high rate of relapse associated with traditional chemotherapy treatments for these tumor cells was not observed in mice injected with siRNA nanoparticles, indicating the potential long-term therapeutic benefit of this highly selective, systemic RNAi approach in the treatment of cancers.

### Vector-Based Small Interfering RNAs Suppress Growth of Human Prostate Tumor In Vivo

Prostate cancer is the most common cancer and the second leading cause of cancer-related deaths among men in Western countries. More men are currently diagnosed at the early stages of prostate cancer and can be effectively treated by surgery or radiation. However, in one third of the patients, the disease will recur and metastatic prostate cancer remains essentially incurable. Whereas significant progress has been made in defining the molecular mechanisms of prostate cancer development, the specific molecular regulatory pathways involved in prostate cancer progression have not been fully characterized. However, targeting of currently known pathways may lead to effective treatments for prostate cancer.

Signal transducers and activators of transcription (STAT) were identified originally as key components of cytokine signaling pathways that regulate gene expression. In mammals, there are seven members of the STAT family. All of them possess a similar modular organization comprised of the following domains: the NH2-terminal, coiled-coil, DNA-binding, SH2, and transactivation domains, which are all important for proper functioning. Constitutive activation of one STAT family member, Stat3, has been shown to play a key role in promoting proliferation, differentiation, antiapoptosis, and cell cycle progression. Persistently active Stat3 and its overexpression have been detected in human prostate cancers and have been suggested to be associated with prostate cancer progression. Aberrantly active Stat3 promotes uncontrolled growth and survival through dysregulation of expression of downstream targeted genes, such as cyclin D1, cyclin D2, c-Myc, and p53, and Bcl-xL, Bcl-2, Mcl-1, and Survivin; these genes influence cell cycle progression or inhibit apoptosis. Stat3 exists in a latent form in the cytoplasm until activated by a wide variety of cell surface receptors via tyrosine phosphorylation, dimerization, and translocation into the nucleus, where it binds to STAT-specific DNA response elements in certain promoters. Constitutive Stat3 signaling represents one of the key molecular events in the multistep process leading to carcinogenesis. Thus, Stat3 may represent a new molecular target for therapeutic intervention of prostate cancer.

Several recent reports show that blockade of Stat3 expression in human cancer cells suppresses proliferation in vitro and tumorigenicity in vivo. The approaches include tyrosine kinase inhibitors, antisense oligonucleotides, decoy oligonucleotides, dominant-negative Stat3 protein, and RNA interference (RNAi). In the RNAi approach, a sequence-specific posttranscriptional gene silencing is achieved through a small interfering RNA (siRNA), a short double-stranded RNA molecule in which one strand is complementary (i.e., antisense) to the target mRNA of a selected gene. RNAi technology is currently being used not only as a powerful tool for analyzing gene function, but also for developing highly specific therapeutics. RNAi has been shown to be effective not only in cultured mammalian cells, but also in vivo. Recently, short hairpin RNAs (shRNA) have proven to be effective both in vitro and in vivo at reducing targeted gene expression. These artificial RNAs are apparently transcribed as hairpin RNA precursors from an RNA polymerase III-based vector containing the U6 or H1 promoters in cultured cells, and are processed to their effective mature siRNA forms by Dicer. shRNAs are inexpensive to deliver on plasmids and are quite stable relative to antisense RNAs.

Signal transducer and activator of transcription 3 (Stat3) is constitutively activated in a variety of cancers and it is a common feature of prostate cancer. Thus, Stat3 represents a promising molecular target for tumor therapy. In Gao, L. et al. 2005 Clin Cancer Res 11:6333-6341), the investigators applied a DNA vector-based Stat3-specific RNA interference approach to block Stat3 signaling and to evaluate the biological consequences of Stat3 down-modulation on tumor growth using a mouse model.

To investigate the therapeutic potential of blocking Stat3 in cancer cells, three small interfering RNAs (siRNA; Stat3-1, Stat3-2, and Stat3-3) specific for different target sites on Stat3 mRNA were designed and used with a DNA vector-based RNA interference approach expressing short hairpin RNAs to knockdown Stat3 expression in human prostate cancer cells in vitro as well as in vivo.

Of the three equivalently expressed siRNAs, only Stat3-3 and Stat3-2, which target the region coding for the SH2 domain and the coiled-coil domain, respectively, strongly suppressed the expression of Stat3 in PC3 and LNCaP cells. The Stat3-1 siRNA, which targeted the DNA-binding domain, exerted no effect on Stat3 expression, indicating that the gene silencing efficiency of siRNA may be dependent on the local structure of Stat3 mRNA. The Stat3 siRNAs down-regulated the expression of Bcl-2 (an antiapoptotic protein), and cyclin D1 and c-Myc (cell growth activators) in prostate cancer cells. Inhibition of Stat3 and its related genes was accompanied by growth suppression and induction of apoptosis in cancer cells in vitro and in tumors implanted in nude mice.

These data indicate that studies that use RNAi to counteract disease processes in vivo are emerging.

### STATs and Gene Regulation

STATs (signal transducers and activators of transcription) are a family of latent cytoplasmic proteins that are activated to participate in gene control when cells encounter various extracellular polypeptides. Biochemical and molecular genetic explorations have defined a single tyrosine phosphorylation site and, in a dimeric partner molecule, a Src homology 2 (SH2) phosphotyrosine-binding domain, a DNA interaction domain, and a number of protein-protein interaction domains (with receptors, other transcription factors, the transcription machinery, and perhaps a tyrosine phosphatase). Seven mammalian STAT genes have been identified.

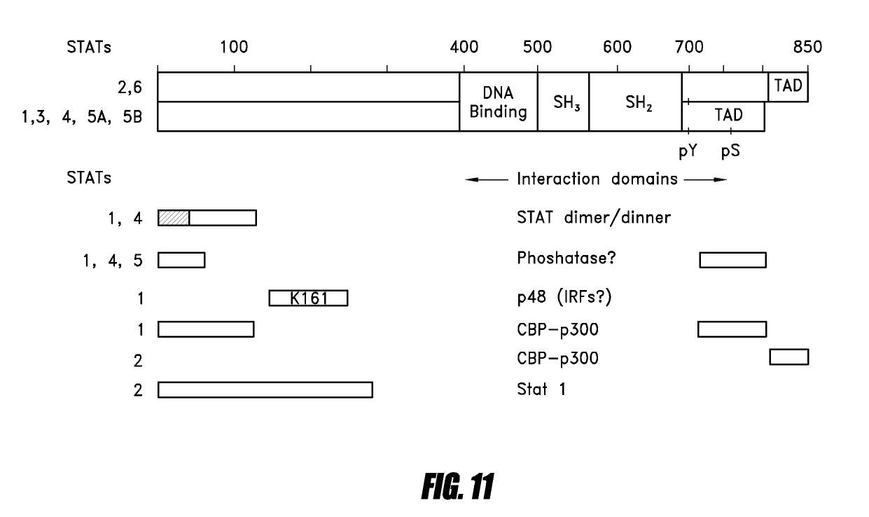

Referring to FIG. 11, the STAT molecules are either ˜850 (Stats 2 and 6) or 750 to 795 amino acids long (Stats 1, 3, 4, 5A, and 5B). The universally shared regions and their boundaries are indicated in the upper panel. Phosphotyrosine (pY) is present in all activated STATs; phosphoserine (pS) is present in activated Stats 1, 3, 4, 5A, and 5B. Transactivation domains (TAD) are shown at the carboxy terminal ends. Protein interaction domains in the STATs listed at the left in the lower panel. The NH₂-terminal (leftmost) domain of Stats 1 and 4 is divided; the dark box indicates that removal of 40 residues of Stat4 destabilizes dimer-dimer interactions in that molecule.

### Example 1

### Construction of siRNA Expression Vectors

A siRNA target located in the SH2 domain of human signal transducer and activator of transcription 3 (Stat3; nucleotides 2144-2162; Genbank accession no. NM_{—}003150) was chosen for use herein based upon our previous study (Gao, L. et al. 2005 Clin Cancer Res 11:6333-6341). The sequence of Stat3-specific hairpin RNA is given as follows: GCAGCAGCTGAACAACATGTTCAAGAGACATGTTGTTCAGCTGCTGCTTTTT. This oligonucleotide contains a sense strand of 20 nucleotides followed by a short spacer (loop sequence: TTCAAGAGA), the antisense strand, and five Ts (terminator). A scrambled siRNA (Ambion) was used as a negative control. Double-stranded DNA oligonucleotides were cloned into pGCsilencerU6/Neo/GFP, which also expresses a green fluorescent protein (GFP) gene (Jikai Chemical, Inc.), to generate plasmids pSi-Stat3 and pSi-Scramble (FIG. 1A).

### Bacteria, Cell Culture, and Stable Cell Line Establishment

The attenuated S. typhimurium phoP/phoQ null strain LH430 was kindly provided by Dr. E. L. Hohmann (Hohmann, E. L. et al. 1996 J Infect Dis 173:1408-1414). This strain was created from S. typhimurium strain SL1344 by deletion of the phoP/phoQ locus (Fu, X. et al. 1992 Int J Cancer 51:989-991). Plasmids were electroporated into Salmonella before use. The mouse prostate cancer cell line RM-1 was obtained from the Shanghai Institute of Cellular Research. The cells were grown in Iscove's modified Dulbecco's medium (Invitrogen) with 10% fetal bovine serum. Cells were co cultured with recombinant bacteria (1×10⁸ cfu) at 37° C. for 30 min. Cell lines were washed and treated first with 100 μg/mL gentamicin to kill all extracellular bacteria and then with 5 μg/mL of tetracycline to prevent intracellular bacterial multiplication. Stable RM-1 clones, containing integrated plasmids, were selected and maintained by treating the cells with 200 μg/mL G418.

### Tumorigenic Assays

C57BL6 mice (n=10 per group) were injected s.c. with the cell lines described above (2×10⁶ cfu) into the upper flank. Tumor development was followed for 60 days. All animal studies were conducted in accordance with the principles and procedures outlined in the NIH Guide for the Care and Use of Laboratory Animals under assurance no. A3873-1.

### Northern and Western Blotting

Cell lysis, protein quantification, and Western blot analyses were carried out as described previously (Gao, L. et al. 2005 Clin Cancer Res 11:6333-6341). Antibodies against Stat3, phosphorylated Tyr⁷⁰⁵-Stat3 (p-Stat3), cyclin D1, c-Myc, VEGF, and antimouse were obtained from Santa Cruz Biotechnology. Antibody against Bcl-2 was obtained from DAKO Biotech. Antibody against Ki-67 was obtained from Biogenex. Protein bands were detected using enhanced chemiluminescence (Amersham). Total RNA (20 μg) and ³²P-labeled cDNAs of Stat3 and actin were used as probes. mRNA level was quantified using a Molecular Dynamics PhosphorImager.

### Cell Cycle, Apoptosis, and Proliferation Assays

Cell cycle phase distribution was determined by flow cytometry. An Annexin V-CY3 apoptosis detection kit (Sigma) was used for detecting apoptosis. Tumor tissue sections from animals were used for H&E staining and terminal deoxynucleotidyl transferase-mediated nick-end labeling (TUNEL) assays, as described previously (Gao, L. et al. 2005 Clin Cancer Res 11:6333-6341). Cell proliferation was assayed using a 3-(4,5-dimethylthiazol-2-yl)-2,5-diphenyltetrazolium bromide (MTT) staining kit (Sigma) as per manufacturer's protocol; the cell growth inhibition rate was calculated as follows: A=(1-absorbance of experimental group/absorbance of control group) X 100%.

Antitumor Activity of Recombinant S. typhimurium on Established Prostate Tumors

RM-1 cells were transplanted into mice s.c. to generate a primary tumor. After the development of a palpable tumor at the site of inoculation, tumors were excised, and the primary tumor fragments (1.5 mm³) were implanted by surgical orthotopic implantation in between two lobes of the prostatic gland in a recipient group of C57BL6 mice according to methods described previously (Fu, X. et al. 1992 Int J Cancer 51:989-991; Hoffman, R. M. 1999 Invest New Drugs 17:343-359). Five days after implantation, mice were divided into three groups (n=10 per group) and injected i.v. with 1×10⁷ cfu of attenuated S. typhimurium carrying different plasmids. One set of mice was sacrificed 18 days after administration of bacteria, and tumors were excised, weighed, and measured diameter. Tumor metastases were counted in the liver, lung, spleen, kidney, and lymph nodes. The remaining mice were followed over 70 days for survival after treatment with different plasmids.

### Analysis of Bacterial Distribution

Tissue samples from the primary tumor, the liver, the spleen, and from other sets of tumor-bearing mice were used for bacterial distribution and clearance studies. Normal and tumor tissues were excised, weighed, minced thoroughly, and homogenized. The diluted tissue homogenates were plated onto Luria-Bertani agar containing ampicillin in triplicate, and the colony count was determined on the next day. The tissues were also observed under a fluorescence microscope to determine the extent of bacterial infection. A portion of the tissues was also prepared for histochemical analyses.

### Gelatin Zymography Assay

The gelatinolytic activities of matrix metalloproteinase-2 (MMP-2) were examined according to the method described previously (Lalu, M. M. et al. 2002 Biochem Biophys Res Commun 296:937-941).

### Data Analyses

The significance of the in vitro and in vivo data was determined using the Student's two-tailed t test. The significance of the differences between median data values was determined using the two-tailed Mann Test. P<0.05 was deemed statistically significant. Data are presented as mean±SD.

TABLE 1
Effect of siRNAs on cell growth and apoptosis in RM-1 cells.

| Group | Apoptotic cells, % | G₀-G₁, % | S, % |
|---|---|---|---|
| (n = 10) | (mean ± SD) | (mean ± SD) | (mean ± SD) |
| Mock | 0.4 ± 0.15 | 43.0 ± 2.02 | 45.7 ± 2.36 |
| pSi-Scramble | 1.3 ± 0.27* | 51.7 ± 2.65 | 36.2 ± 2.93 |
| pSi-Stat3 | 28.9 ± 3.14* | 71.2 ± 2.35* | 3.2 ± 0.35* |

*P < 0.01 versus pSi-Scramble.

TABLE 2
Antitumor effects of bacterially transferred Stat3-specific siRNAs.

| Group | Mean weight (g) | | Mean tumor |
|---|---|---|---|
| (n = 10) | Mouse | Tumor | volume (mm³) |
| Mock | 26.52 ± 3.06 | 3.43 ± 0.89 | 2,458.51 ± 602.18 |
| pSi-Scramble | 25.36 ± 2.58 | 1.45 ± 0.61* | 589.22 380.34* |
| Salmonella alone | 25.00 ± 1.22 | 1.66 ± 0.23* | 585.44 ± 220.21* |
| pSi-Stat3 | 24.31 ± 2.36 | 0.38 ± 0.24^{†} | 216.42 134.15^{†} |

*P < 0.05 versus mock
^{†}P < 0.01 versus mock

TABLE 3
Tumor metastases following siRNA treatment.

| | | | | | | | Total Number |
|---|---|---|---|---|---|---|---|
| | | | | | | Lymph | of metastases |
| Group | Spleen | Liver | Kidney | Lung | Bladder | Nodes | # (%) |
| Mock | 2 | 1 | 1 | 5 | 8 | 8 | 25 (100) |
| pSi- | 0 | 0 | 0 | 2 | 4 | 6 | 12 (48) |
| Scramble | | | | | | | |
| pSi-Stat3 | 0 | 0 | 0 | 1 | 1 | 2 | 4 (16) |

TABLE 4
Salmonella species, subspecies, serotypes, and
their usual habitats, Kauffmann-White scheme

| Salmonella species and | No. of serotypes | |
|---|---|---|
| subspecies | within subspecies | Usual habitat |
| S. enterica subsp. | 1,454 | Warm-blooded animals |
| enterica (I) | | |
| S. enterica subsp. | 489 | Cold-blooded animals |
| salamae (II) | | and the environment ^{a} |
| S. enterica subsp. | 94 | Cold-blooded animals |
| arizonae | | and the environment |
| (IIIa) | | |
| S. enterica subsp. | 324 | Cold-blooded animals |
| diarizonae | | and the environment |
| (IIIb) | | |
| S. enterica subsp. | 70 | Cold-blooded animals |
| houtenae | | and the environment |
| (IV) | | |
| S. enterica subsp. | 12 | Cold-blooded animals |
| indica (VI) | | and the environment |
| S. bongori (V) | 20 | Cold-blooded animals |
| | | and the environment |
| Total | 2,463 | |

^{a} Isolates of all species and subspecies have occurred in humans.

TABLE 5
Salmonella nomenclature in use at CDC, 2000^{a}

| Taxonomic position | Nomenclature |
|---|---|
| Genus (italics) | Salmonella |
| Species (italics) | enterica, which includes subspecies I, II, |
| | IIIa, IIIb, IV, and VI |
| | bongori (formerly subspecies V) |
| Serotype (capitalized, | The first time a serotype is mentioned in the |
| not italicized)^{b} | text; the name should be preceded by the |
| | word “serotype” or “ser.” |
| | Serotypes are named in subspecies I and |
| | designated by antigenic formulae in |
| | subspecies II to IV, and VI and S. bongori |
| | Members of subspecies II, IV, and VI and S. |
| | bongori retain their names if named before |
| | 1966 |

^{a}In 1984 investigators updated the reporting system used at CDC for Salmonella. The major changes that CDC made and that result in a difference from the 1984 reporting system are (i) capitalization of the serotype name, (ii) inclusion of subspecies VI and S. bongori, and (iii) adoption of the type species name S. enterica.
^{b}Examples of serotype designations are Salmonella serotype (ser.) Typhimurium, Salmonella II 50:b:z₆, Salmonella IIIb 60:k:z, and Salmonella ser. Marina (IV 48:g, z₅₁:-).

TABLE 6
Examples of Salmonella nomenclature currently seen in the literature

| Complete name | CDC designation | Other designations |
|---|---|---|
| S. enterica^{a} subsp. enterica | Salmonella ser. Typhi | Salmonella typhi |
| ser. Typhi | | |
| S. enterica^{a} subsp. enterica | S. ser. Typhimurium | Salmonella typhimurium |
| ser. Typhimurium | | |
| S. enterica^{a} subsp. salamae | S. ser. Greenside | S. II 50:z:e,n,x, S. greenside |
| ser. Greenside | | |
| S. enterica^{a} subsp. arizonae | S. IIIa 18:z₄,z₂₃:- | “Arizona hinshawii” ser. |
| ser. 18:z₄,z₂₃:- | | 7a,7b:1,2,5:- |
| S. enterica^{a} subsp. | S. IIIb 60:k:z | “A. hinshawii” ser. 24:29:31 |
| diarizonae ser. 60:k:z | | |
| S. enterica^{a} subsp. houtenae | S. ser. Marina | S. IV 48:g,z₅₁:-, S. marina |
| ser. Marina | | |
| S. bongori ser. Brookfield | S. ser. Brookfield | S. V 66:z₄₁:-, S. brookfield |
| S. enterica^{a} subsp. indica | S. ser. Srinagar | S. VI 11:b:e,n,x, S. srinagar |
| ser. Srinagar | | |

^{a}S. choleraesuis and S. enteritidis are also used.

TABLE 7
Recent Clinical and Preclinical Studies Evaluating
Live, Salmonella Vaccines and Vectors

| Attenuated | | Attenuating | Foreign | Test | | Reactogenicity |
|---|---|---|---|---|---|---|
| vector | Strain | mutations(s) | antigen | species | Route | (humans) |
| Salmonella | Ty21a | galE/rpoS/others | PA | mouse | ip | — |
| typhi | ZH9 | aroC ssaV | LT-B | human | oral | well tolerated |
| | | | HBV- | | | |
| | | | core | | | |
| | BRD1116 | aroAC htrA | F1-V | mouse | in | — |
| Salmonella | 1160 | phoPQ purB | UreAB | human | oral | well tolerated |
| typhimurium | | phoPQ aroA asd | SopE- | primate | ig | — |
| | | | Gag | human | oral | ongoing study |
| | M020 | phoPQ asd | F1-V | mouse | oral | — |
| | | | | rabbit | ig | |
| | VNP-20009 | purI msbB | CD | human | iv | minimal |
| | (Vion | | | | | |
| | Pharmaceuticals | | | | | |
| | Inc.) | | | | | |

CD, cytosine deaminase; HBV, hepatitis B virus; ig, intragastric; in, intranasal; ip, intraperitoneal; iv, intravenous; LT-B, heat-labile toxin B; PA, protective antigen.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. An attenuated Salmonella sp. comprising a short hairpin (sh) RNA construct.

2. The attenuated Salmonella sp. of claim 1, wherein the Salmonella sp. is Salmonella typhi or Salmonella typhimurium.

3. The attenuated Salmonella sp. of claim 2, wherein the Salmonella sp. is Salmonella typhi.

4. The attenuated Salmonella sp. of claim 2, wherein the Salmonella sp. is Salmonella typhimurium.

5. The attenuated Salmonella sp. of claim 1, wherein the shRNA has a complimentary sequence to an oncogene.

6. The attenuated Salmonella sp. of claim 1, wherein the shRNA has a complimentary sequence to a gene that is overexpressed in cancer cells.

7. The attenuated Salmonella sp. of claim 1, wherein the shRNA has a complimentary sequence to a gene that encodes a cytoplasmic protein that promotes the survival of a human tumor.

8. The attenuated Salmonella sp. of claim 7, wherein the shRNA has a complimentary sequence to Stat3.

9. The attenuated Salmonella sp. of claim 7, wherein the tumor is selected from the group consisting of lung cancer, liver cancer, kidney cancer, breast cancer, and prostate cancer.

10. The attenuated Salmonella sp. of claim 9, wherein the tumor is prostate cancer.

11. A method of inhibiting the growth or reducing the volume of a solid tumor cancer comprising administering an effective amount of an attenuated Salmonella sp. to a patient having a solid tumor cancer, wherein said attenuated Salmonella sp. expresses a short hairpin (sh) RNA construct.

12. The method of claim 11 wherein the Salmonella sp. is Salmonella typhi or Salmonella typhimurium.

13. The method of claim 12, wherein the Salmonella sp. is Salmonella typhi.

14. The method of claim 12, wherein the Salmonella sp. is Salmonella typhimurium.

15. The method of claim 11, wherein the shRNA has a complimentary sequence to an oncogene.

16. The method of claim 1, wherein the shRNA has a complimentary sequence to a gene that is overexpressed in cancer cells.

17. The method of claim 1, wherein the shRNA has a complimentary sequence to a gene that encodes a cytoplasmic protein that promotes the survival of a human tumor.

18. The method of claim 17, wherein the shRNA has a complimentary sequence to Stat3.

19. The method of claim 17, wherein the tumor is selected from the group consisting of lung cancer, liver cancer, kidney cancer, breast cancer, and prostate cancer.

20. The method of claim 19, wherein the tumor is prostate cancer.
